(19) United States
(12) Patent Application Publication
(10) Pub. No.: US 2009/0304625 A1
(43) Pub. Date: Dec. 10, 2009

(54) MODULATORS OF CARDIAC CELL HYPERTROPHY AND HYPERPLASIA

(76) Inventors: Ahsan Husain, Hoover, AL (US); Nawazish Naqvi, Birmingham, AL (US); Robert M. Graham, Darlinghurst (AU); Louis J. Dell'talia, Homewood, AL (US); Ming Li, Birmingham, AL (US)

Correspondence Address: FISH & RICHARDSON P.C., P.O BOX 1022, Minneapolis, MN, 55440-1022, US

(21) Appl. No.: 12/161,311

(22) PCT Filed: Jan. 18, 2007
(86) PCT No.: PCT/US07/60703

§ 371 Date: Oct. 27, 2008

Related U.S. Application Data
(60) Provisional application No. 60/759,737, filed on Jan. 18, 2006.

(51) Int. Cl. A61K 31/496; A61K 48/00; A61K 39/395; G01N 33/53; A61K 38/18; A61K 38/20; A61P 9/00

(52) U.S. Cl. 424/85.2; 514/252.18; 514/44 R; 424/173.1; 435/7.21; 514/12

(57) ABSTRACT

Provided are compositions and methods for modulating cardiac cell hypertrophy and hyperplasia using inhibitors of c-Kit activity.

### CROSS-REFERENCE TO RELATED APPLICATIONS

The present application claims priority to U.S. Ser. No. 60/759,737 filed Jan. 18, 2006.

### STATEMENT REGARDING FEDERALLY SPONSORED RESEARCH

This invention was made with government, support under Grant No. R01HL79040 and Grant No. P50HL077100 awarded by the NIH. The government has certain rights in the invention.

### BACKGROUND OF THE INVENTION

Soon after birth cardiomyocytes irreversibly exit the cell cycle and, thereafter, hyperplastic growth is not evident (R. A. Poolman, et al. (1999); H. Oh et al., (2001); K. B. S. Pasumarthi, L. J. Field (2002)). Hypertrophic growth, characterized by an increase in cardiomyocyte size, is an adaptive response of the adult heart to pathological stresses that increase workload, such as hypertension. Cardiac enlargement initially facilitates cardiac performance by normalizing systolic wall stress, but eventually results in impaired myocardial oxygenation and apoptotic cell loss, leading to cardiac dysfunction, arrhythmias and sudden death.

### BRIEF SUMMARY OF THE INVENTION

The methods and compositions described herein provides a means to avoid problems associated with hypertension using agents that inhibit c-Kit activity. More specifically, provided herein is a method of inhibiting hypertension-induced hypertrophy of cardiac cells, comprising contacting the cardiac cells with an inhibitor of c-Kit activity. This method can he performed in vitro or in vivo, for example by administering to the subject a therapeutic amount of an inhibitor of c-Kit activity.

A method of screening for agents that inhibit hypertension-induced hypertrophy of cardiac cell's is disclosed herein. The screening steps comprise contacting the cardiac cells with the agent to be tested and measuring c-Kit activity. A decrease in c-Kit activity as compared to a control indicates feat the agent inhibits hypertension-induced hypertrophy of the cardiac cells.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 shows reduced mortality in Kit^{w}/Kit^{w-v}-SAC (suprarenal aortic constriction) mice. FIG. 1A is a graph of Kaplan-Meier survival plots that show lower survival rates for wildtype (WT) mice after SAC (broken line, n=24) than for sham-operated WT mice (solid line, n=9, P=0.028 by log-rank test). FIG. 1B shows that survival rates for Kit^{w}/Kit^{w-v} mice after SAC (broken line, n=23) are similar to those for sham-operated mice (solid line, n=10).

FIG. 2 shows left ventricle (LV) cardiomyocyte hypertrophy and reduced cell density in Kit^{w}/Kit^{w-v} mice in comparison to controls despite similar SAC-induced hypertension, LV enlargement and atrial natriuretic peptide (ANP) expression. FIG. 2A shows mean arterial blood pressures. FIG. 2B shows cardiac ANP mRNA expression. FIG. 2C shows LV weight/body weight ratios. FIG. 2D shows LV cardiomyocyte cross-sectional area. FIG. 2E shows LV cardiomyocyte density, in WT-sham, WT-SAC, Kit^{w}/Kit^{w-v}-sham and Kit^{w}/Kit^{w-v}-SAC mice, n=5-7 per group. Values are means±SEM. *P<0.05, **P<0.01, and ***P<0.001 for intra-genotype comparisons and †P<0.05 and ††P<0.01 for inter-genotype comparisons.

FIG. 3 shows SAC produces similar increases in BrdU⁺ LV cardiac interstitial cells, collagen I and collagen III expression, and fibrosis in WT and Kit^{w}/Kit^{w-v} mice. FIG. 3A shows time-dependent increases in BrdU⁺ cardiac interstitial cells in WT and Kit^{w}/Kit^{w-v} mice after SAC. FIG. 3B shows LV myocardium BrdU⁺ cardiac interstitial cells (arrowheads) surrounded by vimentin and located between cardiomyocytes containing myosin heavy chain. FIG. 3C shows time-dependent changes in collagen I mRNA levels. FIG. 3D shows time-dependent changes in collagen III mRNA levels. FIG. 3E shows time-dependent changes in LV interstitial fibrosis in WT and Kit^{w}/Kit^{w-v} mice after SAC. n=5 per group in WT-SAC, Kit^{w}/Kit^{w-v}-SAC. WT-sham and Kit^{w}/Kit^{w-v}-sham mice. Values are means±SEM. *P<0.05, **P<0.01, and ***P<0.001 for intra-genotype comparisons. Bar=20 μm.

FIG. 4 shows SAC induces proliferation of LV cardiomyocytes in vivo in adult Kit^{w}/Kit^{w-v} mice. FIG. 4A shows SAC-induced changes in Ki67⁺ LV cardiomyocyte density. FIG. 4B shows SAC-induced changes in BrdU⁺ LV cardiomyocyte density. Localization in adult LV cardiomyocytes of Kit^{w}/Kit^{w-v} mice with SAC of BrdU, myosin heavy chain and vimentin (C-E); Ki67 and myosin heavy chain (F-H), and H3P, BrdU and myosin heavy chain (I, J). Arrows in ‘J’ indicate sites of apparent cell division. SAC-induced expression of cyclins D1 (K), D2 (L), and D3 (M), and p21^{waf1/cip1} (N), p27^{kip}1 (O) mRNA in the LV after 7 days of SAC or sham operation in WT and Kit^{w}/Kit^{w-v} mice. Values are means±SEM. *P<0.05 and **P<0.01 for intragenotype comparisons and †P<0.05 for inter-genotype comparisons. (P) Positive correlation between the Ki67⁺-LV cardiomyocyte density and velocity of circumferential shortening (VCFr) in Kit^{w}/Kit^{w-v} mice after 7 and 14 days of SAC. Panels C, D, E, and I have the same magnification and panels F, G, H have the same magnification. Bars=20 μm.

FIG. 5 shows SAC induces changes in vimentin expression and localization in the heart of Kit^{w}/Kit^{w-v} and WT mice. (A) Cardiac vimentin mRNA levels in Kit^{w}/Kit^{w-v} and WT mice after 3, 7, and 14 days of SAC or a sham operation. Values are means±SEM. *P<0.05 and **P<0.01 for ultra-genotype comparisons. Localization of vimentin nd myosin heavy chain in the LV myocardium after 14 days of a sham operation in WT (B) or Kit^{w}/Kit^{w-v} (C) mice or after 14 days of SAC in WT (D) or Kit^{w}/Kit^{w-v} (E) mice. Arrowheads in ‘E’ indicate the localization of vimentin at cardiomyocyte intercalated discs. B-E are at the same magnification. Bar=20 μm.

FIG. 6 shows that c-kit tyrosine kinase dysfunction increases hypertension-dependent expansion of c-kit⁺ CSCs. FIG. 6A shows that SAC produced an increase in c-kit⁺ CSCs in WT and W/W^{v} LVs. But, at 7 days of SAC, this expansion was increased ≈5.5-fold in W/W^{v} LVs compared to WT LVs (P<0.01). Basal levels of c-kit⁺ CSCs after sham operation were similar in WT and W/W^{v} LVs. Values are means±s.e.m. n=5 per group. *P<0.05, **P<0.01 and ***P<0.001 for intra-genotype comparisons and †\P<0.01 for inter-genotype comparisons. These comparisons were made using ANOVA followed by Tukey's test. FIG. 6B shows that in W/W^{v} mice subjected to 7 or 14 days of SAC, LV c-kit⁺ CSC numbers were positively associated (r=0.85, P=0.018) with systolic LV function (VCFr).

FIG. 7 shows that SAC produces similar increases in fibroblast (BrdU+/vimentin+LV cardiac interstitial cells) proliferation and fibrosis in WT and W/W^{v} mice. FIG. 7A shows timedependent changes in BrdU+/vimentin+LV cardiac interstitial cells in WT and W/W^{v} mice after SAC. FIG. 7B shows time-dependent changes in LV interstitial fibrosis in WT and W/W^{v} mice after SAC. Five μm hearts sections were stained with Picric Acid Sirius Red F3BA. Using 30 to 40 digitized images collected by the video camera, we determined the volume percent collagen of each medium power field in a blinded manner. The volume percent collagen in WT-sham group at all time points was 0-2%. Volume percent collagen >2% in a medium power field was considered as a field with fibrosis. The results were expressed as the percentage of total medium power fields with fibrosis. Values are means±SEM. n=5 animals per group. *P <0.05, **P<0.01, and ***P<0.001 for intra-genotype comparisons.

FIG. 8 shows c-kit protein expression in cardiomyocytes adjacent to large c-kit⁺ CSC clusters. There was a ˜17-fold greater abundance of c-kit⁺ cardiomyocytes adjacent to large c-kit⁺ CSC clusters than those adjacent to isolated (1-2 cells) c-kit⁺ CSCs. Values are means±s.e.m. 26 CSC clusters from five 7-day-SAC W/W^{v} LVs and 17 isolated CSCs from five 7-day-SAC W/W^{v} LVs were analyzed and P was determined using Student's t-test. ***P<0.001.

FIG. 9 shows that actuarial survival is worse in WT mice after SAC than in W/W^{v} mice, n=24 for WT mice and n=21 for W/W-v mice.

### DETAILED DESCRIPTION OF THE INVENTION

The disclosed method and compositions may be understood more readily by reference to the following detailed description of particular embodiments and the Examples included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

The adult mammalian heart typically responds to pressure overload with cardiomyocyte hypertrophy, but not proliferation. Therefore, heart failure is usually the ultimate outcome. The present methods address tills problem by providing uses for c-Kit inhibitors and means of screening for c-Kit inhibitors. Also provided is a method of inducing dedifferentiation and/or subsequent proliferation of cardiomyocytes.

More specifically, provided herein is a method of inhibiting hypertension-induced hypertrophy of a cardiac cell or plurality of cardiac cells in vivo or in vitro. The method comprises the step of contacting the cardiac cell(s) with a therapeutically effective amount of an inhibitor of c-Kit activity. Preferably, the cardiac cells express c-kit.

Inhibit, inhibiting, and inhibition mean to decrease an activity, response, condition, disease, or other biological parameter. This can include but is not limited to tire complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

Hypertrophy refers to an enlargement or overgrowth of an organ or part of the body due to the increased size of the constituent cells. Hypertrophy occurs in the skeletal muscle and cardiac muscle because, of increased work. Cardiac hypertrophy is recognizable microscopically by the increased size of the cells. In contrast, hyperplasia refers to an increase in the size of a tissue or organ due to an increase in the number of constituent cells.

The herein disclosed compositions and methods rescue cardiac cells from stresses such as hypertension and inhibit cardiomyocyte hypertrophy by promoting proliferation of tire constituent cells rather than hypertrophy. Soon after birth cardiomyocytes irreversibly exit the cell cycle and, thereafter, hyperplastic growth is not evident (R. A. Poolmam et al. (1999); H. Oh et al., (2001); K. B. S. Pasumarthi, (2002)). Differentiated adult cardiomyocytes have long been considered incapable of cell division. However, the present application provides a method of stimulating dedifferentiation and proliferation of differentiated cardiomyocytes. As used herein cardiac cells refers to adult cardiomyocytes, cardiac stem cells, dedifferentiated adult cardiomyocytes and fused cardiomyocte/cardiac stem cells. A cardiac stem cell as used herein refers to cells that are capable of differentiating into cardiac progenitor cells such as, for example, cardiomyocytes. Cardiac cells and cardiac stem cells as used herein do not include bone marrow precursor cells.

Blood pressure is the result of two farces, one created by the heart as it pumps blood into the arteries and the other created by the arterial blood vessels as they exert resistance to the blood flow from the heart. Hypertension, or elevated blood pressure, indicates that the heart is working harder than normal, putting both the heart and the arteries under a greater strain. If high blood pressure is not treated, the heart may have to work progressively harder to pump enough blood and oxygen to the body's organs and tissues to meet their needs. Cardiac hypertrophy is thought to be a structural adaptation of the heart, at least in part, as a compensatory mechanism for increased blood pressure and wail stress (i.e., increased mechanical load). The herein, provided methods inhibit this compensatory mechanism, at least in part, by promoting cardiomyocyte proliferation as substitute compensation.

The receptor tyrosine kinase (RTK) c-Kit (stem cell factor receptor (SCFR); CD117) is a member of the class III family of RTKs, characterized by an extracellular ligand binding region containing 5 immunoglobulin repeats, a hydrophobic transmembrane domain, and an intracellular kinase domain split by an insert. The ligand for the c-Kit receptor has now been identified, molecularly cloned and expressed (Yarden et. al., The EMBO Journal, 6, 3341-3351 (1987)). The encoded protein, known as stem cell factor (SCF), mast cell growth factor (MGF), or steel factor (SLF) is the product of a gene which resides at the steel (S1) locus. Binding of SCF to c-Kit initiates a signal transduction cascade that includes receptor autophosphorylation and subsequent phosphorylation on numerous intracellular substrates.

Provided herein is a methods comprising use of inhibitors of c-Kit activity. The inhibitor can be any c-Kit inhibitor, including, for example, Imatinib mesylate. Imatinib mesylate (formerly STI571, [GLEEVEC®]; Novartis Pharmaceuticals Corporation, East Hanover, N.J.) is a selective inhibitor for the Abelson tyrosine kinase (Ab1) and platelet-derived growth factor tyrosine kinases (Buehdunger E., et al. Cancer Res., 56: 100-104, 1996). Imatinib mesylate also inhibits the c-Kit receptor tyrosine kinase (Krystal G. W., et al. Clin, Cancer Res., 6: 3319-3326, 2000; Buehdunger E., et al. J. Pharmacol Exp. Ther., 295: 139-145, 2000).

Other examples of c-Kit inhibitors include, for example, SU5416 and SU6668. SU5416 and SU6668 are small-molecule inhibitors of RTKs such as Flk-1 (VEGF-R2; KDR) mat have structural and sequence similarity to c-Kit. SU5416 is a more selective and potent inhibitor of the Flk-1 receptor. In contrast, SU6668 exhibits a broader inhibitory target profile, with effects on platelet-derived growth factor (PDGF) receptor and fibroblast growth factor (FGF) receptor in addition to Flk-1. Both compounds have been shown to be selective for other tyrosine kinases, for example, with inhibitory concentration of 50% (IC₅₀) above 10 μM tor epidermal growth factor (EGF) receptor, Src, Met, and ZAP-70. In cell-based and preclinical animal models, both compounds have also been shown to exhibit antiangiogenic properties. SU5416 inhibits vascular endothelial growth factor (VEGF)-induced and SU6668 VEGF- and FGF-induced proliferation of human umbilical vein endothelial cells in culture. However, neither compound potently inhibits the growth of tumor cells grown in culture. In addition, both compounds inhibit the growth of a variety of tumor cells grown as subcutaneous xenografts in athymic mice. Furthermore, SU6668 causes regression of established xenograft tumors in mice. Intravital fluoresence videomicroscopy in mouse tumor xenograft models shows that SU5416 and SU6668 also inhibit tumor angiogenesis in vivo. However, in contrast to the anti-mitogenic properties described for SU5416 and SU6668, disclosed herein are proliferation promoting effects of these molecules in the context of hypertensive cardiac cells.

The c-Kit inhibitor of the provided methods can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, microRNA molecules, short interfering RNAs (siRNAs) and external guide sequences. The functional nucleic acid molecules can act as effectors, inhibitors, modulators, and stimulators of a specific activity possessed by a target-molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA, genomic DNA, or polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using dimethylsulfate (DMS) and diethyl pyrocarbonate (DEPC). It is preferred that antisense molecules bind the target molecule with a dissociation constant (k_{d})less than or equal to 10⁻⁶, 10⁻⁸, 10−10, or 10⁻¹². A representative sample of methods and techniques which aid in the design and use of antisense molecules can be found in, for example, U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437. Antisense oligonucleotides to c-Kit are disclosed in U.S. Pat. No. 5,989,849, which is hereby incorporated herein by reference in its entirety for this teaching.

Aptamers are molecules mat interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with dissociation constants from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target, molecules can be found in, for example, U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermoleculary. Ribozymes are thus catalytic nucleic acid molecules. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes (see, for example, but not limited to, U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 98/58058 by Ludwig and Sproat, WO 98/58057 by Ludwig and Sproat and WO 97/18312 by Ludwig and Sproat); hairpin ribozymes (see, for example, but not limited to U.S. Pat. Nos. 5,631,115, 5,646,031, 5,683,902, 5,712,384, 5,856,188, 5,866,701, 5,869,339, and 6,022,962); and tetrahymena ribozymes (see, for example, but not limited to U.S. Pat. Nos. 5,595,873 and 5,652,107). There are also a number of ribozymes that are not found in natural systems, but which have been engineered to catalyze specific reactions de novo (see, for example, but not limited to U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in, for example, U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid molecules. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in, for example, U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that, bind a target nucleic acid molecule forming a complex, which is recognized by RNase P. RNaseP then cleaves the target, molecule. EGSs can be designed to specifically target a RNA molecule of choice. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)). Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992): WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in, for example, U.S. Pat. Nos. 5,168,053, 5,624,824, 5,683,873, 5,728,521, 5,869,248, and 5,877,162.

Gene expression can also be effectively silenced In a highly specific manner through RNA interference (RNAi). This silencing was originally observed with the addition of double stranded RNA (dsRNA) (Fire, A., et al. (1998) Nature, 391, 806 811; Napoli, C., et al. (1990) Plant Cell 2, 279 289; Hannon, G. J. (2002) Nature, 418, 244 251). Once dsRNA enters a cell, it is cleaved by an RNase III-like enzyme. Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409, 363 366; Hammond, S. M., et al. (2000) Nature, 404:293-296). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309 321). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-574). However, the effect of RNAi or siRNA or their use is not limited to anytype of mechanism.

Short interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498; Ui-Tei, K., et al. (2000) FEBS Lett 479:79-82). siRNA can be chemically or in vitro-synthesized or can lie the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands), siRNA can also be synthesized in vitro using kits such as Amnion's SILENCER® (Ambion, Austin Tex.) siRNA Construction Kit. Disclosed herein are any siRNA designed as described above based on the sequences for c-Kit or SCF. The nucleic acid and amino acid sequences for c-Kit and SCF are known and can be found on the GenBank database. The Accession numbers for c-Kit include, but are not limited to AAH52457 (mouse), AAH71593 (human) and BAA02094 (rat). The Accession numbers for SCF include, but are not limited to, P21583 (human), P20826 (mouse) and NP_{—}001012477 (rat). In addition, siRNAs for silencing gene expression of c-Kit are commercially available (SURESILENCING™ Human c-Kit siRNA; Zymed Laboratories, San Francisco, Calif.).

The production of siRNA from a vector is more commonly done through the transcription of a short hairpin RNAs (shRNAs). Kits for the production of vectors comprising shRNA are available, such as, for example, Imgenex's GENESUPPRESSOR™ (Imgenex Corporation, San Diego, Calif.) Construction Kits and Invitrogen's BLOCK-IT™ (Invitrogen, Carlsbad, Calif.) inducible RNAi plasmid and lentivirus vectors. Disclosed herein are any shRNA designed as described above based on the sequences for the herein disclosed inflammatory mediators.

Optimally, the inhibitor of the provided methods can block the binding of stem cell factor (SCF) to c-Kit. Methods for inhibiting the binding of a protein to its receptor can, for example, be based on the use of molecules that compete for the binding site of either the ligand or the receptor.

Thus, the inhibitor can be, for example, a polypeptide that competes for the binding of a receptor without activating the receptor. Likewise, a ligand binding inhibitor can be a decoy receptor that competes for the binding of ligand. Such a decoy receptor can be a soluble receptor (e.g., lacking transmembrane domain) or it can be a mutant receptor expressed in a cell but lacking the ability to transduce a signal (e.g., lacking cytoplasmic tail). Optimally, the inhibitor is naturally produced by a subject. Alternatively, the inhibitory molecule can be designed based on targeted mutations of either the receptor or the ligand. Thus, as an illustrative example, the inhibitor is a fragment of SCF, wherein the fragment is capable of binding c-Kit without activating the receptor. The ligand binding inhibitor optimally is a polypeptide comprising a fragment of c-Kit. The c-Kit fragment optimally lacks the cytoplasmic tail or the transmembrane domain.

Antibodies specific for either a ligand or a receptor can also be used to inhibit binding. The antibody optimally is specific c-Kit. For example, c-Kit neutralizing antibodies are commercially available such as anti-rhSCFR (Boehringer-Ingelheim, Germany). Optimally, the antibody is specific for SCF. The term antibodies is used herein in a broad sense and includes both polyclonal and monoclonal antibodies. In addition to intact immunoglobulin molecules, fragments, chimeras, or polymers of immunoglobulin molecules are also useful in the methods taught herein, as long as they are chosen for their ability to interact with SCF or c-Kit such that SCF is inhibited from interacting with c-Kit. The antibodies can be tested for their desired activity using the in vitro assays, or by analogous methods, after which their in vivo therapeutic or prophylactic activities are tested according to known clinical testing methods.

The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain's is identical with or homologous to corresponding sequences in antibodies derived from another species or belonging to another antibody class or subclass, as well as fragments of such antibodies, as long as they exhibit the desired antagonistic activity (See. U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include, site-specific mutagenesis of the nucleic acid encoding the antibody or antibody fragment. (Zoller, M. J. Curr. Opin. Biotechnol. 3:348-354, 1992).

As used herein, the term antibody or antibodies can also refer to a human antibody and/or a humanized antibody. Many non-human antibodies (e.g., those derived from mice, rats, or rabbits) are naturally antigenic in humans and thus can give rise to undesirable immune responses when administered to humans. Therefore, the use of human or humanized antibodies in the methods serves to lessen the chance that an antibody administered to a human will evoke an undesirable immune response.

Examples of techniques for human monoclonal antibody production are known in the art and include those described by Boerner et al. (J. Immunol., 147(1):86-95, 1991). Human antibodies (and fragments thereof) can also be produced using phage display libraries (Hoogenboom et al., J. Mol. Biol., 227:381, 1991: Marks et al. J. Mol. Biol., 222:581, 1991).

The disclosed human antibodies can also be obtained from transgenic animals. For example, transgenic, mutant mice that are capable of producing a foil repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen-binding portion of an antibody.

Methods for humanizing non-human antibodies are well known in the art. For example, humanized antibodies can be generated according to the methods described in Jones et al., Nature, 321:522-525 (1986), Riechmann et al., Nature, 332:323-327 (1988), and Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

The antibodies to c-Kit or SCF described herein can be administered using a variety of techniques including, for example, those described herein. Nucleic acid approaches for antibody delivery also exist. The broadly neutralizing c-Kit antibodies and antibody fragments can be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by a variety of means including, for example, those described herein.

The c-Kit inhibitor of the provided methods can induce proliferation of the cardiac cells. Thus, provided is a method of increasing cardiac cell proliferation, comprising contacting the cardiac cell with a c-Kit inhibitor. Preferably, the cardiac cells are cardiac stem cells. Titus, provided is a method of increasing cardiac stem cell numbers, comprising contacting a cardiac stem cell with an inhibitor of c-kit activity.

The c-Kit inhibitor of the provided methods can also improve contractility of the cardiac cell. Thus, provided is a method of improving cardiac cell contractility, comprising contacting the cardiac cell with a c-Kit inhibitor. The cardiac cells of the provided methods optimally have been, are, or will be subject to stress, such as increased work load in response to increased blood pressure in the heart.

Also provided herein is a method of reducing or inhibiting hypertrophic cardiomyopathy in a subject, comprising administering to the subject a therapeutic amount of an inhibitor of c-Kit activity. The hypertrophic cardiomyopathy can be hypertension-induced. The provided method can reduce or prevent the incidence of hypertrophic cardiomyopathy in the subject. Thus, the method can reduce the mortality (i.e., delay death) or improve the morbidity (i.e, reduce or delay one or more symptoms or signs associated with cardiomyopathy) of the subject. By prevent is meant a reduction or delay in clinical symptoms or signs.

The c-Kit inhibitor of the provided methods can be used to identify cytokines that inhibit hypertension-induced hypertrophy or increase cardiac cell number. Thus, provided is a method of identifying cytokines that are associated with inhibition hypertension-induced hypertrophy (e.g., cytokines that inhibit hypertrophy), comprising contacting cardiac cells wife an inhibitor of c-kit activity; and detecting changes in cytokine expression or activity. An increase in cytokine expression or activity as compared to control indicates feat the cytokine is associated with inhibition of hypertension-induced hypertrophy. As used herein, detecting changes in cytokine expression refers to detecting mRNA levels (e.g., via Northern blot analysis or RT-PCR) or protein levels (e.g., via ELISA or Western blot). Methods of detecting changes in expression are known in the art. Cytokines identified by the method can be administered to subjects in need or can be contacted wife cardiac cells to increase proliferation of stem cells and the like. Cytokines can be combined with c-kit inhibitors in the methods described herein.

As used herein, control refers to cardiac cells feat have not be contacted with an inhibitor of c-kit activity. Also provided are methods of inhibiting hypertension-induced hypertrophy in a subject, comprising administering to the subject a cytokine. A method of increasing cardiac cell numbers, comprising contacting a cardiac cell with a cytokine. Preferably, the cytokine is selected from the group consisting of insulin-like growth factor-1, interlukin-6, bone morphogenic protein-1, and chemokine (C-C motif) ligand 2 (CCL2).

Hypertrophic Cardiomyopathy (HCM) is a cardiac disorder with heterogeneous expression, unique pathophysiology, and a diverse clinical course, for which several disease-causing mutations in the genes encoding proteins of the cardiac sacomere have been reported. The main feature of hypertrophic cardiomyopathy is an excessive thickening of the heart muscle. Thickening is seen in the ventricular septal measurement (normal range 0.08-1.2 mm), and in weight. In HCM, septal measurements may be in the range of 1.3 mm to 6.0 mm. Heart muscle may also thicken in normal individuals as a result of high blood pressure or prolonged athletic training.

As used herein, subject includes a vertebrate, more specifically a mammal (e.g., a human, horse, pig, rabbit, dog, sheep, goat, non-human primate, cow, cat, guinea pig or rodent), a fish, a bird or a reptile or an amphibian. Subjects include adult and newborn subjects, as well as fetuses. As used herein, “patient” or “subject” may be used interchangeably and can refer to a subject afflicted with a disease or disorder. Thus, the term subject includes human and veterinary subjects.

The subject of the provided methods can be hypertensive. The subject cart have mild hypertension (Stage 1). The subject can have moderate hypertension (Stage 2). The subject can have severe hypertension (Stage 3). The subject can have very severe hypertension (Stage 4). Exemplary human blood pressure ranges are provided in Table 1.

TABLE 1

| | |
|---|---|
| | Ranges for Most Adults |
| Blood Pressure Category | (systolic/diastolic) |

Blood Pressure Ranges

| | |
|---|---|
| Optimal Blood Pressure | Systolic below 120 mm Hg |
| (systolic/diastolic) | Diastolic below 80 mm Hg |
| Normal Blood Pressure | Systolic 120 to 130 mm Hg |
| | Diastolic 80 to 85 mm Hg |
| High Normal Blood Pressure | Systolic 130 to 139 mm Hg |
| | Diastolic 85 to 89 mm Hg |
| Hypertension (High Blood Pressure) | Systolic above 140 mm Hg |
| | Diastolic above 90 mm Hg |
| Mild Hypertension (Stage 1) | Systolic 140 to 159 mm Hg |
| | Diastolic 90 to 99 mm Hg |
| Moderate Hypertension (Stage 2) | Systolic 160 to 179 mm Hg |
| | Diastolic 100 to 109 mm Hg |
| Severe Hypertension (Stage 3) | Systolic 180 to 209 mm Hg |
| | Diastolic 110 to 119 mm Hg |
| Very Severe Hypertension (Stage 4) | Systolic greater than 210 mm Hg |
| | Diastolic greater than 120 mm Hg |

Blood Pressure in Children
A child's blood pressure is normally much lower than an adult's.
Children are at risk for hypertension if they exceed the following levels:

| | |
|---|---|
| * Ages three to five | 116/76 |
| * Ages six to nine | 122/78 |
| * Ages 10 to 12 | 126/82 |
| * Ages 13 to 15 | 136/86 |

Note:
If one measurement is normal and the other elevated, the higher category of either measurement is usually used to determine severity. For example, if systolic pressure is 165 (moderate) and diastolic is 92 (mild), the patient would still be diagnosed with moderate hypertension. It should be strongly noted that a high systolic pressure compared to a normal or low diastolic pressure should be a major focus of concern in most adults.

Also provided is a method of inducing dedifferentiation and proliferation of an adult cardiomyocyte, comprising contacting the cardiomyocyte with an inhibitor of c-Kit activity. Dedifferentiation refers to the regression of a specialized cell or tissue to a simpler, more embryonic, unspecialized form. As disclosed herein, the provided compositions and methods induce cardiomyocytes to regress to a more embryonic form in order to re-initiate cell division and, thus, to proliferate.

Also provided is a method of screening for agents that inhibit hypertension-induced hypertrophy of cardiac cells, comprising contacting the cardiac cells or cardiac stem cells with the agent to be tested and measuring c-Kit activity. A decrease in c-Kit activity as compared to a control indicates an agent that inhibits hypertension-induced hypertrophy.

Methods for evaluating c-Kit activity are known in the art. For example, c-Kit activity can be measured by detecting phosphotyrosine residues in the cytoplasmic domain of c-Kit. For example, c-Kit [pYpY568/570], [pY703], [pY721], [pY730], [pY823] and [pY936] phospho-specific antibodies are commercially available (BioSource, Camarillo, Calif.). Initially, SCF binding to the extracellular domain of c-Kit markedly increases the intrinsic kinase activity by stimulating autophosphorylation of tyrosine 823, leading to phosphorylation of multiple tyrosine residues in the cytoplasmic domain. Cytoplasmic proteins then bind to the phosphotyrosine sites to initiate a range of downstream signaling pathways. Documented signaling/adapter protein interactions with c-Kit phosphotyrosine sites include: Grb2 with pY703 and pY936; Grb7 with pY936; PI3-K with pY719; PLCg with pY730; and multiple signaling and adapter proteins (protein tyrosine phosphatases SHP1 and SHP2, Src family kinases Fyn and Lyn, and Chk) with pYpY568/570. These interactions induce proliferation, apoptosis, adhesion, and migration and appear to be cell type specific. Thus, c-Kit activity can also be measure by detecting the association of c-Kit to the adapter proteins.

Methods of screening for agents that inhibit c-Kit activity and hypertension-induced hypertrophy of a cardiomyocyte or cardiac cells are provided. The method comprises contacting a cardiac stem cell with an agent to be tested, measuring c-Kit activity, wherein a decrease in c-Kit activity as compared to a control indicates that the agent inhibits hypertension-induced hypertrophy of cardiac cells. In general, agents that inhibit c-Kit activity and hypertension-induced hypertrophy of a cardiomyocyte may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedures) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, e.g., Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known, in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect, on c-Kit activity should be employed whenever possible.

When a crude extract is found to have a desired activity, further fractionation of the positive lead extract is necessary to isolate chemical constituents responsible for the observed, effect. Thus, the goal of the extraction, fractionation, and purification process is the careful characterization and identification of a chemical entity within the crude extract having an activity inhibits c-Kit activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art.

The disclosed compositions can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. Thus, the disclosed compositions can be administered orally, parenterally (e.g., intravenously), by intramuscular injection, by intraperitoneal injection, transdermally, extracorporeally, topically or the like, including topical intranasal administration or administration by inhalant.

Parenteral administration of the composition, if used, is generally characterized by injection. Injectables can be prepared in conventional forms, either as liquid solutions or suspensions, solid forms suitable, for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein in its entirety for the release system.

The materials may be in solution, suspension (for example, incorporated into microparticles, liposomes, or cells). These may be targeted to a particular cell type via antibodies, receptors, or receptor ligands. The following references are examples of the use of this technology to target proteins to specific cell types (Senter, et al., Bioconjugate Chem., 2:447-451, (1991); Bagshawe, K. D. Br. J. Cancer, 60:275-281, (1989); Bagshawe, et al., Br. J. Cancer, 58:700-703, (1988); Senter, et al., Bioconjugate Chem., 4:3-9, (1993); Battelii, et al. Cancer Immunol. Immunother., 35: 421-425, (1992); Pietersz and McKenzie, Immunolog. Reviews, 129:57-80, (1992); and Roffler, et al., Biochem. Pharmacol, 42:2062-2065, (1991)).

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishes (such as those based on Ringer's dextrose), and the like. Preservatives and other additives may also be present such as, for example, antimicrobials, antioxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids or binders may be desirable,

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As disclosed above, the provided methods can comprise the administration and uptake of exogenous DNA into the cells of a subject (i.e., gene transduction or transfection). The disclosed nucleic acids can be in the form of naked DNA or RNA, or the nucleic acids can be in a vector for delivering the nucleic acids to the cells, whereby the antibody-encoding DNA fragment is under me transcriptional regulation of a promoter, as would be well understood by one of ordinary skill in die art. The vector can be a commercially available preparation, such as an adenovirus vector (Quantum Biotechnologies, Inc., Laval, Quebec, Canada). Delivery of the nucleic acid or vector to cells can be via a variety of mechanisms. As one example, delivery can be via a liposome, using commercially available liposome preparations such as LIPOFECTIN®, LIPOFECTAMINE™ (GIBCO-BRL, Inc., Gaithersburg, Md.), SUPERFECT® (Qiagen, Inc. Hilden, Germany) and TRANSFECTAM® (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION™ machine (ImaRx Pharmaceutical Corp., Tucson, Ariz.).

As one example, vector delivery can be via a viral system, such as a retroviral vector system that can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding the desired MT-MMP inhibitor (or active fragment thereof). The exact method of introducing the altered nucleic acid into mammalian cells is, of course, not limited to the use of retroviral vectors. Other techniques are widely available for this procedure including die use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

Effective dosages and schedules for administering the compositions can be determined empirically, and making such determinations is within the skill in the art. The dosage ranges for the administration of the compositions are those large enough to produce the desired effect in which the symptom's disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary and can, for example, be administered in one or more dose administrations daily, for one or several days. Guidance can be found in the literature for appropriate dosages for given classes of pharmaceutical products. A typical daily dosage of the provided compositions used alone might range from about 1 μg/kg to up to 100 mg/kg of body weight or more per day, depending on the factors mentioned above.

Following administration of a disclosed composition for treating, inhibiting, or preventing cardiomyocyte hypertrophy, the efficacy of the therapeutic composition can be assessed in various ways well known to the skilled practitioner. For instance, one of ordinary skill in the art will be able to determine if a composition is efficacious in treating or inhibiting hypertrophic cardiomyopathy in a subject using an electrocardiogram (ECG/EKG), echocardiogram (ECHO), or MRI.

An ECG records the electrical signals from the heart and is performed by placing electrodes on the chest, wrist and ankles. In hypertrophic cardiomyopathy, the ECG usually shows an abnormal electrical signal due to muscle thickening and disorganization of the muscle structure. In a minority of patients (approximately 10%) the ECG may be normal or show only minor changes. ECG abnormalities are also not specific to hypertrophic cardiomyopathy and may be found in other heart conditions.

An ECHO produces a picture of the heart such that excessive thickness of the muscle can be easily measured. Additional equipment called “Doppler” ultrasound can produce a color image of blood flow within the heart and measure the heart's contraction and filling. Turbulent flow can be detected. Therefore ECHO provides a very thorough assessment of hypertrophic cardiomyopathy.

As MRI can provide tomographic high resolution pictures of the heart, it has recently become an important new test well suited for the assessment of the size and extent of left ventricular hypertrophy in HCM. In fact, recent studies have shown that a cardiac MRI may be better than an echocardiogram to reliably detect hypertrophy in areas such as the left ventricular anterolateral wall and apex. As a result, in some patients an echocardiogram may not be sufficient to confidently exclude a diagnosis of HCM and in that situation a cardiac MRI may be recommended. In addition, because of its high spatial resolution, a cardiac MRI may also be performed to define the precise extent of wall thickening.

The compositions disclosed herein to perform the disclosed methods can be made using any method known to those of skill in the art for that particular reagent or compound unless otherwise specifically noted. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see, for example, Sambrook et al., Molecular Cloning: A Laboratory Manual, 2nd Edition (Cold Spring Harbor Laboratory Press, Cold Spring Harbor, N.Y., 1989) Chapters 5,6) to purely synthetic methods, for example, by the cyanoethyl phosphoramidite method using a Milligen or Beckman System 1Plus DNA synthesizer (for example. Model 8700 automated synthesizer of Milligen-Biosearch, Burlington, Mass. or ABI Model 380B). Synthetic methods useful for making oligonucleotides are also described by Ikuta et al., Ann. Rev. Biochem 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug, Chem. 5:3-7 (1994).

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor ate discussed, each and every combination and permutation of inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-P, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D, This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms a, an, and the include plural reference unless the context clearly dictates otherwise. Thus, for example, reference to an inhibitor includes a plurality of such inhibitors, reference to the inhibitor is a reference to one or more inhibitors and equivalents thereof known to those skilled in the art, and so forth.

Optional or optionally means that the subsequently described event, circumstance, or material may or may not occur or be present, and that the description includes instances where the event, circumstance, or material occurs or is present and instances where it does not occur or is not present.

Ranges may be expressed herein as from about one particular value, and/or to about another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will foe further understood that the endpoints of each of the ranges are significant both in relation to the other endpoint, and independently of the other endpoint unless the context specifically indicates otherwise. Finally, it should be understood that all of the individual values and sub-ranges of values contained within, an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge, the accuracy and pertinency of the cited documents. It will be clearly understood that, although a number of publications are referred to herein, such reference does not constitute an admission that any of these documents forms part of the common general knowledge in the art.

Throughout the description and claims of this specification, the word comprise and variations of the word, such as comprising and comprises, means including but not limited to, and is not intended to exclude, for example, other additives, components, integers or steps.

### EXAMPLES

The following examples are put forth so as to provide those of ordinary skill in the art with a complete disclosure, and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in C or is at ambient temperature, and pressure is at or near atmospheric.

### Example 1

### Effect of c-Kit Tyrosine Kinase Dysfunction on Cardiomyocytes

As used herein, W/W^{v} mice and Kit^{w}/Kit^{w-v} mice are used interchangeably.

### Materials and Methods

Animals; Male WBB6F1/J-Kit^{w}/Kit^{w-v} (Kit^{w}/Kit^{w-v}) mice and their wild type littermates (WT) (S. J. Galli, et al. (1987)) were purchased from the Jackson Laboratory (Bar Harbor, Me.). Animals were given drinking wafer and food ad libitum and handled according to National Institutes of Health and University of Alabama at Birmingham institutional animal care and use committee guidelines. In the Kit^{w}/Kit^{w-v} mice, the c-kit W allele has a deletion in its transmembrane domain and has the characteristics of a null mutation while the c-kit W^{v} allele is a point mutation wherein the kinase domain of c-kit has markedly diminished but detectable kinase activity.

Induction of pressure overload: At eight weeks of age, WT or Kit^{w}/Kit^{w-v} mice were subjected to suprarenal aortic constriction (SAC) to induce hypertension and, thus, pressure overload, or to sham operation, as previously described (M. Li et al. (2004)). One group (both SAC and sham-operated animals) was allowed to recover from surgery for 2 days and animals surviving this peri-operative period were then followed for survival over the next 28 days. A second group were sacrificed at 3, 7 or 14 days after SAC, and a third group was treated with the mast cell stabilizer, sodium chromoglycate (60 mg/kg/day, intraperitoneal, osmotic minipump) for one week before and then for the 7 days after surgery, at which time they were sacrificed.

Hemodynamic measurement; Cardiac hemodynamics were determined immediately before sacrifice by micromanometry, as previously described (G. J. Perry et al. (2001)). Briefly, after induction of anesthesia with isofluorane (˜1-2%) a 1.4 F high fidelity pressure transducer (SPR-671, Millar instruments, Houston, Tex.) was passed via the right carotid artery into the left ventricle (LV) of the heart. Electrodes were attached to allow ECG and heart rate recordings. LV pressure, ECG and heart rate were monitored until stable recordings were obtained. The pressure transducer was then slowly withdrawn into fee aorta for measurement of central arterial pressure (M. Li et al. (2004)).

Echocardiographic evaluations of cardiac function: Echocardiography was performed on lightly anesthetized mice (isoflurane (Abbot Laboratories, Denmark) in oxygen) using a Sonos 5500 (Phillips, Bothell, Wash.) cardiac ultrasound system, as previously described (G. J. Perry et al. (2001)). LV dimensions were obtained from parasternal long-axis long axis views by two-dimensional-guided M-mode imaging. A cursor was positioned perpendicular to the interventricular septum and posterior wall of the LV at the level of the papillary muscles and an M-mode image was obtained at a sweep speed of 100 mm/s and used to determine diastolic and systolic LV wall thickness. LV end-diasiolic dimensions (LVDD) and LV end-systolic chamber dimensions (LVSD). Ejection time (EjT) and RR intervale were obtained by pulsed doppler of LV out-flow at the aortic valve level. Systolic function was calculated from LV dimensions as fractional shortening (FS), and the rate-corrected velocity of circumferential shortening (VCFr) as follows: FS=(LVDD LVSD)/LVDD and VCFr=FS/(EjT−RR^{0.5}) (S3). Recording of echocardiographic images was performed in random order with respect to the SAC or sham treatment animals. We were unable to blind for genotype because of distinctive coat coloring for Kit^{w}/Kit^{w-v} versus WT mice. However, determination of chamber dimensions was blinded for all groups by storing the echocardiographic images using generic file names and making the measurements several days after the images were obtained.

mRNAs expression in the mouse heart: This was determined by real time quantitative RT-PCR, as detailed previously (M. Li et al. (2004)). The primers sets for cyclins D1, D2, and D3, and ANP, p21^{waf1/cip1}, p27^{kip}, collagen I collagen III, vimentin, and GAPDH, and their PGR product sizes are shown in Table 2. All sequences in Table 2 are shown in the 5′ to 3′ direction.

TABLE 2
Primer sets

| | | | | |
|---|---|---|---|---|
| cyclin D1 | 5′ primer | AGGAGCAGAAGTGCGAAGAGGA | SEQ ID NO: 1 | 490 bp |
| | 3′ primer | AAAGTGCGTTGTGCGGTAGC | SEQ ID NO: 2 | |
| cyclin D2 | 5′ primer | CCCTGTACACTCGAACCGTTAT | SEQ ID NO: 3 | 478 bp |
| | 3′ primer | AGTAGAAGCCCAAATTCACCAA | SEQ ID NO: 4 | |
| cyclin D3 | 5′ primer | GTAAAATCCACACACCAGCATTT | SEQ ID NO: 5 | 497 bp |
| | 3′ primer | CTAACCCTGCTCTGATGAAGATG | SEQ ID NO: 6 | |
| p21^{waf1/cip1} | 5′ primer | CTGCAAGAGAAAACCCTGAAGT | SEQ ID NO: 7 | 492 bp |
| | 3′ primer | AGGAGACCCCAAAGTCCTACTC | SEQ ID NO: 8 | |
| p27^{kip1} | 5′ primer | TTCAGATGAGCCGCCTGGATTT | SEQ ID NO: 9 | 499 bp |
| | 3′ primer | TTAACAAGTGGGCAATTTTGTG | SEQ ID NO: 10 | |
| ANP | 5′ primer | CCTGTGTACAGTGCGGTGTC | SEQ ID NO: 11 | 455 bp |
| | 3′ primer | ACACACCACAAGGGCTTAGG | SEQ ID NO: 12 | |
| collagen I | 5′ primer | ACGGCTGCACGAGTCACAC | SEQ ID NO: 13 | 514 bp |
| | 3′ primer | GGCAGGCGGGAGGTCTT | SEQ ID NO: 14 | |
| collagen | 5′ primer | GTTCTAGAGGATGGCTGTACT | SEQ ID NO: 15 | 514 bp |
| III | | AAACACA | | |
| | 3′ primer | TTGCCTTGCGTGTTTGATATTC | SEQ ID NO: 16 | |
| vimentin | 5′ primer | GTCCAAGTTTGCTGACCTCTCT | SEQ ID NO: 17 | 568 bp |
| | 3′ primer | TTCTTGCTGGTACTGCACTGTT | SEQ ID NO: 18 | |
| GAPDH | 5′ primer | ATGGTGAAGGTCGGTGTG | SEQ ID NO: 19 | 633 bp |
| | 3′ primer | ACCAGTGGATGCAGGGAT | SEQ ID NO: 20 | |

Immunohistochemistry and confocal microscopy: To evaluate cell proliferation, mice were given an intraperitoneal injection of BrdU (30 mg/kg body weight; Roche, Nutley, N.J.) 12 hours before sacrifice. Multiple antibodies and stains were applied as previously reported (D. Orlic et al. (2001 a); D. Orlic et al., (2001b)). Briefly, mouse hearts were immersion-fixed in 4% paraformaldehyde and stored in 70% ethanol until paraffin embedding and sectioning. Sections (5 μm) were mounted on slides, deparaffinized in xylene and rehydrated in ethanol. Tissue sections were treated with the Avidin/Biotin Blocking Kit (SP-2001, Vector Laboratories, Burlingame, Calif.), followed with the Mouse on Mouse (M.O.M.) Immunodetection Kit, Fluorescein (FMK-2201, Vector Laboratories, Burlingame, Calif.) in conjunction with heavy chain cardiac myosin (MHC) mouse monoclonal antibody (1:50; ab-15, Abeam, United Kingdom). Sections were blocked with 5% goat serum in 1% bovine serum for 1 hour at room temperature. Primary antibodies (final concentration): Ki67 rabbit monoclonal (RM9106-S, Lab Vision Corporation, Fremont, Calif.) (1:50); BrdU rat monoclonal (6326, Abeam, United Kingdom (1:50); phosphohistone-3 rabbit polyconal (065701, Upstate, Charlottesville, Va.) (1:200); vimentin rabbit polyclonal (7783, Abeam, United Kingdom) (1:150); laminin chicken polyclonal (14055, Abeam, United Kingdom) (1:50) were combined in an appropriate volume of 5% goat serum, and applied to sections by overnight incubation at 4° C. The sections were incubated with ALEXA FLUOR® 350 goat anti-rat (blue), ALEXA FLUOR® 488 goat anti-chicken (green) and ALEXA FLUOR® 594 goat anti-rabbit (red) to visualize the specific stains. All secondary antibodies were from Molecular Probes, Eugene, OR. Image acquisition was performed on a Leica DM6000B epifluorescence microscope (Leica Microsystems, Bannockburn, Ill.) with a Hamamatsu ORCA ER cooled CCD camera and SimplePCI software (Compix, Inc., Cranberry Township, Pa.). To determine which cell type the nuclei of interest were located in, different focal planes were examined by detailed deconvolution or by laser confocal microscopy (Leica DMIRBE inverted Nomarski/epifluorescence microscope outfitted with Leica TCS NT Laser Confocal software) to ensure acquisition of the correct image. Images were adjusted appropriately to remove background fluorescence.

For cardiomyocyte cross-sectional area measurements, heart, tissue was embedded in OCT compound, frozen in methylbutane with liquid nitrogen, and kept at −80° C. until sectioned. Frozen sections (5 μm) were fixed in cold acetone for 10 minutes and dried at RT for 1 hour. Sections were stained with laminin (IMMH-7, Laminin Immunohistology Kit, Sigma-Aldrich, St. Louis, Mo.) to outline the basement membrane of cardiomyocytes (D. E. Vatner et al. (2000)). This procedure used a biotinylated secondary antibody and EXTRAVIDIN® (Sigma-Aldrich, St. Louis, Mo.) peroxidase and AEC chromogen for colorization. Images of tissue in cross-sectional orientation were acquired (40× objective) in a blinded manner, the total field size measured, and myocytes were counted within the field to determine the average myocyte crosssectional area.

For apoptosis analysis, tissue was examined using a terminal dUTP nick end-labeling (TUNEL) kit (Roche, Germany) as reported previously (A. Frustaci et al. (2000)). In brief, heart tissue was fixed in 4% paraformaldehyde and stored in 70% ethanol until paraffin embedding and sectioning. Sections (5 μm) were mounted on slides, deparaffinized in xylene and rehydrated in ethanol. Sections were stained (In situ Cell Death Detection Kit, Fluorescein, Roche, Germany) for the detection and quantification of apoptotic cells. For identification of cardiomyocytes, both anti MHC and anti-laminin antibodies, which outline the different cell shapes, were used. ALEXA FLUOR® (Molecular Probes, Eugene, Oreg.) 594 goat anti-rabbit antibody was used to label the laminin in the basement membrane. For quantification, the TUNEL-positive cells were counted in an entire cardiac section, and the TUNEL-positive interstitial cell or myocytes/mm² was calculated for that sample.

For capillary density determination, frozen sections (5 μm) were fixed in cold acetone for 10 minutes and dried at room temperature for 1 hour. Sections were incubated with Griffonia (Bandeiraea) simplicifolia isolectin B4 (GSL-I-B4, Vector Laboratories, Burlingame, Calif.), which specifically stains endothelial, cells (K. Wakasugi et al. (2002); D. P. Hyink et al. (1996)), followed by a second incubation with ABComplex. The capillaries were visualized by DAB supplemented with 0.3% hydrogen peroxide. Images of tissue in cross-sectional orientation were acquired (40× objective), total field size was measured, and capillaries were counted within the field to determine capillary density.

Collagen analysis: Hearts sections (5 μm) were stained with Picric Acid Sirius Red F3BA as reported (D. E. Vatner et al. (2000)). Quantitative analysis of collagen deposition was accomplished by light microscopy with a video-based image-analyzer system. Collagen volume percent was quantitatively evaluated at medium power (20× objective, 600× video-screen magnification) for interstitial collagen. The LV free wall myocardium was examined by use of PASR-stained sections. A 540-nm (green) filter was used to provide contrast between collagen and the background. Using digitized images collected by the video camera, we determined the volume percent collagen of 30 to 40 randomly selected fields in each section, and the mean value was calculated for each animal. All morphometric measurements were performed in a blinded mariner.

Statistics: Data are presented as mean±SEM. Statistical analysis was performed using the unpaired Student's t test or Tukey's test after ANOVA indicated significant differences. Mortality was analyzed using the Survival LogRank Test, and correlation between Ki67⁺ or BrdU⁺ cardiomyocyte density and VCFr was determined using Pearson Product Moment Correlation coefficent, P values less than 0.05 were considered significant.

### Results

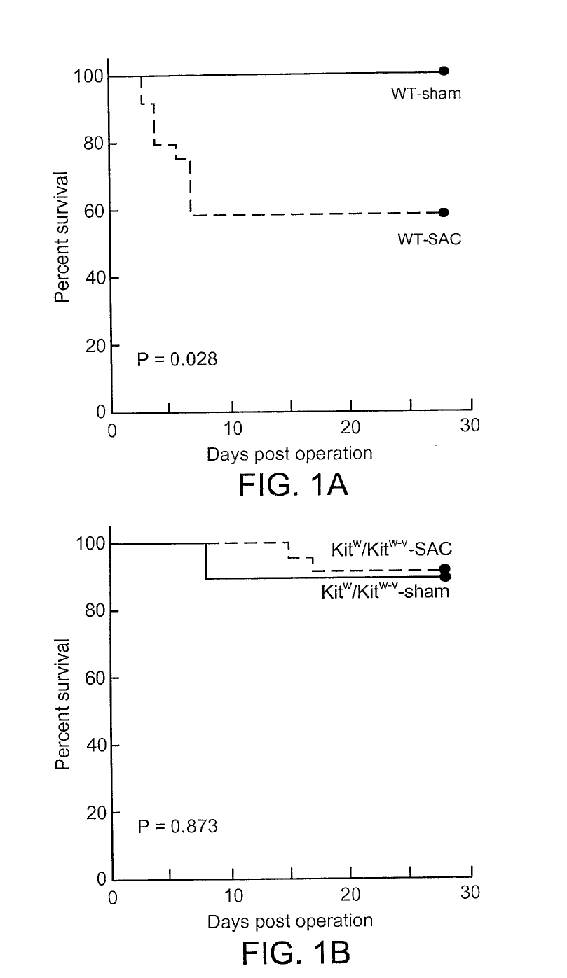

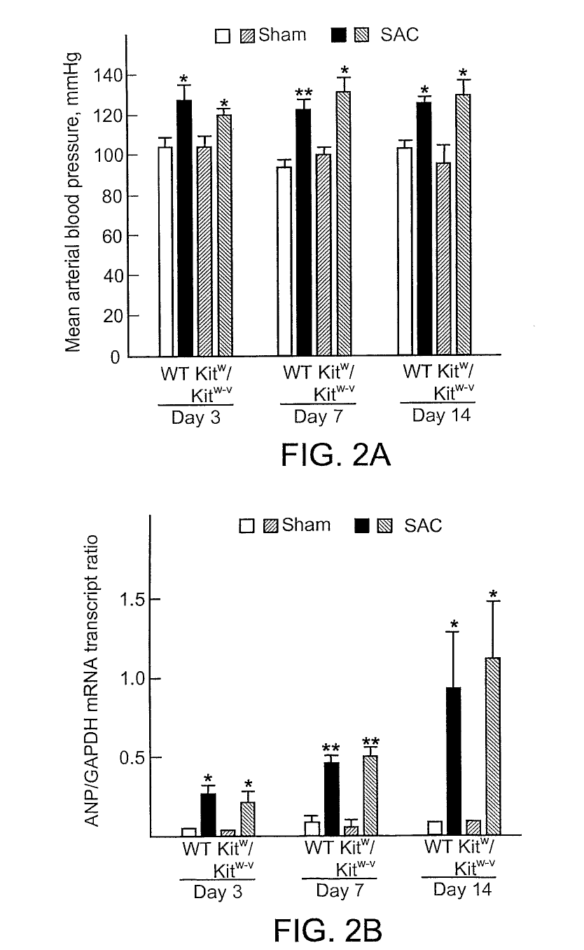

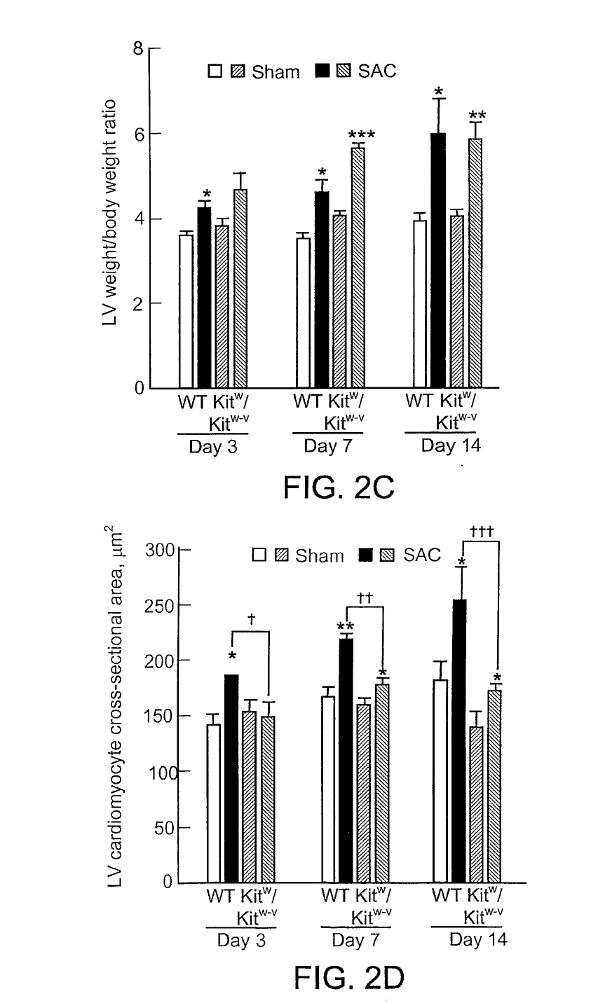

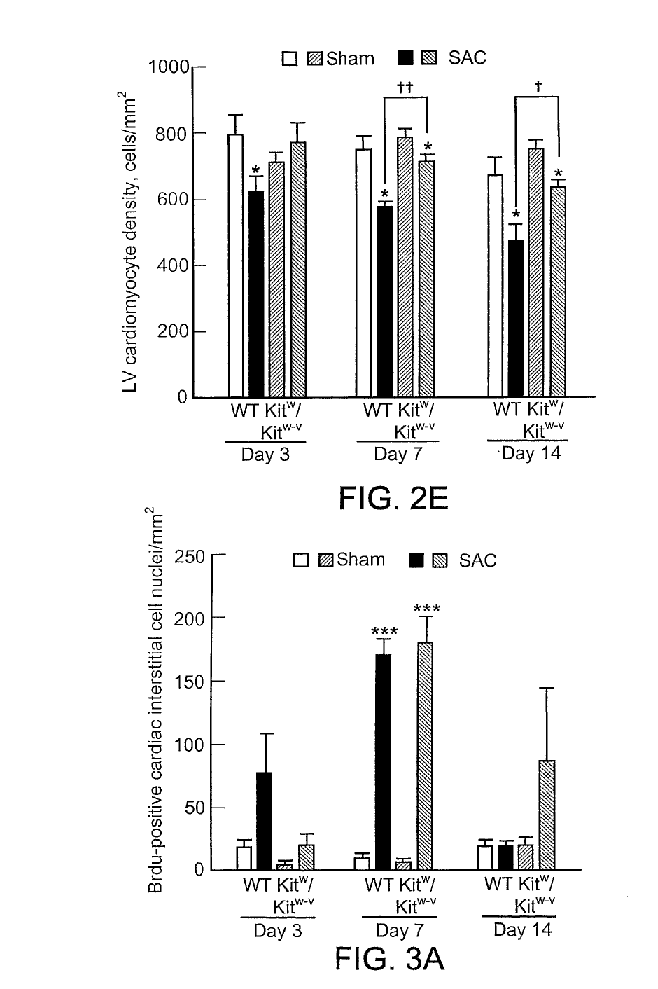

The survival of Kit^{w}/Kit^{w-v} mice and their congenic WT littermates were studies after the induction of hypertension by suprarenal aortic constriction (SAC). In the first 7 days after SAC, 41% of the congenic WT mice died, while there were no deaths in Kit^{w}/Kit^{w-v} mice (FIGS. 1, A and B). Micromanometry and ultrasonography was then used in separate groups of Kit^{w}/Kit^{w-v} and WT mice to assess SAC-induced changes in left ventricular (LV) hemodynamics and function (Table 3). After SAC, WT and Kit^{w}/Kit^{w-v} mice showed similar increases in mean arterial blood pressure, LV atrial natriuretic peptide expression (a marker of re-activation of a fetal gene program observed with hypertrophy), and LV weight/body weight ratio (FIG. 2, A-C). In WT mice, LV enlargement was due to the development of robust concentric hypertrophy, as evidenced by increased LV wall thickness/diameter (Table 3) and increased cardiomyocyte cross-sectional area (FIG. 2D), within 3 days of SAC. This progressed quickly to eccentric LV hypertrophy, similar to that seen in hypertensive humans, in spite of further increases in cardiomyocyte cross-sectional area (FIG. 2D) and LV end-systolic wall stress, and contractility remained unchanged (Table 3). In contrast, Kit^{w}Kit^{w-v} mice showed minimal cardiomyocyte hypertrophy with little change in cardiomyocyte cross-sectional area (FIG. 2D), and no change in systolic wall stress (Table 3). Rather their LV hypertrophy was mainly due to cell proliferation as evidenced by increased cardiomyocyte density shown in FIG. 2E. At 7 days post-SAC, at a time when WT mice were experiencing increased mortality, their LV contractility was enhanced as evident by an approximately 36% higher rate-corrected velocity of circumferential shortening (VCFr) (Table 3). This is significant because hypercontractile cardiac function, reduces early mortality after acute pressure overload (X.-L Du et al., (2004)). Capillary density did not differ in WT- and Kit^{w}/Kit^{w-v}-SAC mice (1,610±37 capillaries/mm² in WT-SAC LV versus 1,750±73 capillaries/mm² in Kit^{w}/Kit^{w-v}-SAC LV). Nevertheless, tissue oxygenation is likely to have been impaired in the WT-, but not Kit^{w}/Kit^{w-v}-SAC hearts, since increased cardiomyocyte diameter is expected to increase diffusion distance (D. Hilfiker-Kleiner et al., (2005)). Although cardiomyocyte apoptosis was not evident in either the WTSAC or Kit^{w}/Kit^{w-v}-SAC mice (no TUNEL-positive cells were found over the entire LV section of each mouse heart), the adverse changes in LV chamber geometry in the WT-SAC hearts, in the face of increasing LV cardiomyocyte hypertrophy, could provide the arrhythmogenic substrate that predisposes to sudden death.

TABLE 3
Ultrasonographic and micromanometric measurements.

| | | | | | |
|---|---|---|---|---|---|
| | | | | | LV wall |
| HR | IVS | PW | LVEDD | LVESD | thickness/ |
| (bpm) | (mm) | (mm) | (mm) | (mm) | diameter |

Postoperative day 3

| | | | | | | |
|---|---|---|---|---|---|---|
| WT-sham | 515 ± 23 | 0.66 ± 0.05 | 0.54 ± 0.05 | 3.73 ± 0.13 | 2.25 ± 0.13 | 0.15 ± 0.02 |
| WT-SAC | 496 ± 14 | 0.92 ± 0.55** | 0.87 ± 0.05** | 3.70 ± 0.18 | 2.13 ± 0.15 | 0.24 ± 0.01** |
| Kitw/Kitw-v-sham | 500 ± 25 | 0.60 ± 0.05 | 0.52 ± 0.05 | 4.01 ± 0.16 | 2.12 ± 0.17 | 0.13 ± 0.01 |
| Kitw/Kitw-v-SAC | 505 ± 19 | 0.66 ± 0.06 | 0.62 ± 0.04 | 4.12 ± 0.22 | 2.55 ± 0.27 | 0.15 ± 0.02 |

Postoperative day 7

| | | | | | | |
|---|---|---|---|---|---|---|
| WT-sham | 439 ± 37 | 0.60 ± 0.04 | 0.57 ± 0.06 | 3.92 ± 0.12 | 2.09 ± 0.18 | 0.15 ± 0.02 |
| WT-SAC | 454 ± 22 | 0.82 ± 0.04* | 0.72 ± 0.05 | 3.93 ± 0.17 | 2.22 ± 0.18 | 0.19 ± 0.02 |
| Kitw/Kitw-v-sham | 521 ± 13 | 0.71 ± 0.05 | 0.63 ± 0.05 | 3.86 ± 0.10 | 1.96 ± 0.08 | 0.16 ± 0.01 |
| Kitw/Kitw-v-SAC | 506 ± 23 | 0.89 ± 0.08 | 0.88 ± 0.09 | 4.09 ± 0.28 | 2.06 ± 0.23 | 0.22 ± 0.02 |

Postoperative day 14

| | | | | | | |
|---|---|---|---|---|---|---|
| WT-sham | 515 ± 24 | 0.75 ± 0.02 | 0.71 ± 0.04 | 3.83 ± 0.16 | 1.94 ± 0.25 | 0.19 ± 0.02 |
| WT-SAC | 420 ± 56 | 0.75 ± 0.01 | 0.66 ± 0.01 | 4.32 ± 0.38 | 2.45 ± 0.45 | 0.15 ± 0.02 |
| Kitw/Kitw-v-sham | 468 ± 8 | 0.71 ± 0.05 | 0.63 ± 0.05 | 4.23 ± 0.24 | 2.22 ± 0.13 | 0.15 ± 0.02 |
| Kitw/Kitw-v-SAC | 446 ± 20 | 0.83 ± 0.05 | 0.81 ± 0.06* | 4.39 ± 0.27 | 2.46 ± 0.32 | 0.19 ± 0.03 |

| | | Systolic | | |
|---|---|---|---|---|
| | VCFr | WS | +dP/dt | −dP/dt |
| | (s-0.5) | (mmHg) | (mmHg/s) | (mmHg/s) |
| Postoperative day 3 | | | | |
| WT-sham | 10.4 ± 0.79 | 79 ± 9 | 10,602 ± 896 | −9,956 ± 7.47 |
| WT-SAC | 12.4 ± 1.33 | 62 ± 8 | 12,395 ± 679 | −9,744 ± 562 |
| Kitw/Kitw-v-sham | 11.4 ± 1.17 | 66 ± 9 | 9,921 ± 1,250 | −8,230 ± 932 |
| Kitw/Kitw-v-SAC | 10.3 ± 1.13 | 64 ± 19 | 13,118 ± 791 | −10,487 ± 1,513 |
| Postoperative day 7 | | | | |
| WT-sham | 10.1 ± 1.12 | 66 ± 13 | 9,373 ± 1,848 | −7,957 ± 1,476 |
| WT-SAC | 11.7 ± 1.72 | 71 ± 10 | 11,538 ± 908 | −9,183 ± 526 |
| Kitw/Kitw-v-sham | 11.9 ± 0.38 | 46 ± 17 | 11,661 ± 3,122 | −9,368 ± 2,111 |
| Kitw/Kitw-v-SAC | 15.9 ± 1.00 | 55 ± 10 | 13,014 ± 596 | −9,305 ± 1,575 |
| Postoperative day 14 | | | | |
| WT-sham | 13.4 ± 2.02 | 49 ± 11 | 13,945 ± 616 | −10,680 ± 602 |
| WT-SAC | 12.5 ± 3.22 | 61 ± 11 | 13,096 ± 2,121 | −8,796 ± 1,497 |
| Kitw/Kitw-v-sham | 10.5 ± 0.60 | 53 ± 8 | 10,188 ± 1,669 | −7,785 ± 927 |
| Kitw/Kitw-v-SAC | 11.9 ± 1.01 | 72 ± 12 | 10,494 ± 1,770 | −7,986 ± 839 |

HR, heart rate:
IVS, interventricular septum thickness at diastole;
PW, posterior wall thickness at diastole;
LVEDD, LV end-diastolic dimension;
LVESD, LV endsystolic dimension;
VCFr, rate-corrected velocity of circumferential shortening:
Systolic WS, LV end-systolic wall stress;
SAC, suprarenal aortic constriction.
*P < 0.05,
**P < 0.01 sham versus SAC within genotype comparisons using unpaired Students t test.
Values are mean ± SEM, n = 4-7/group.

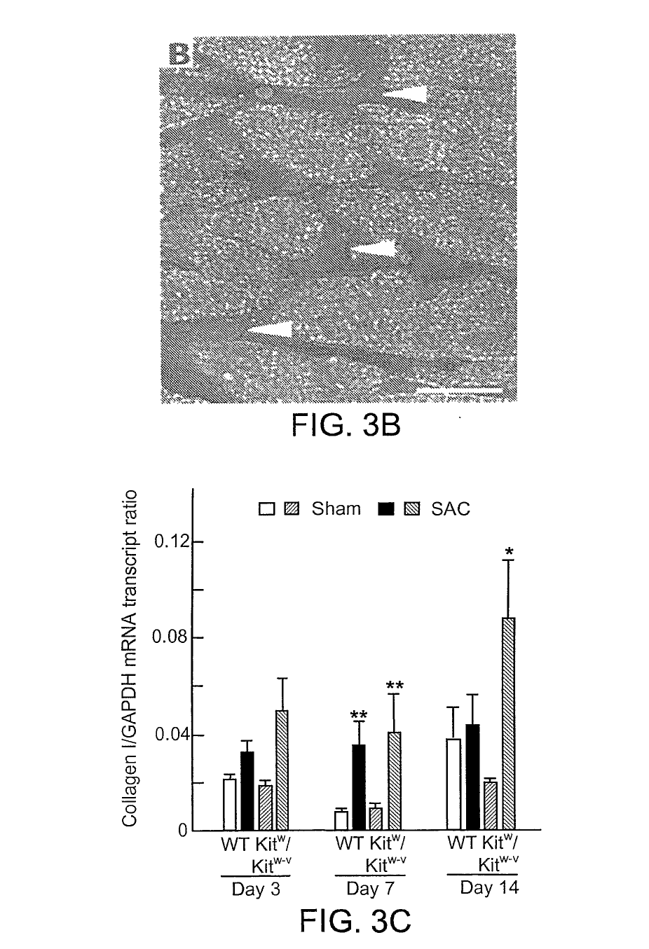

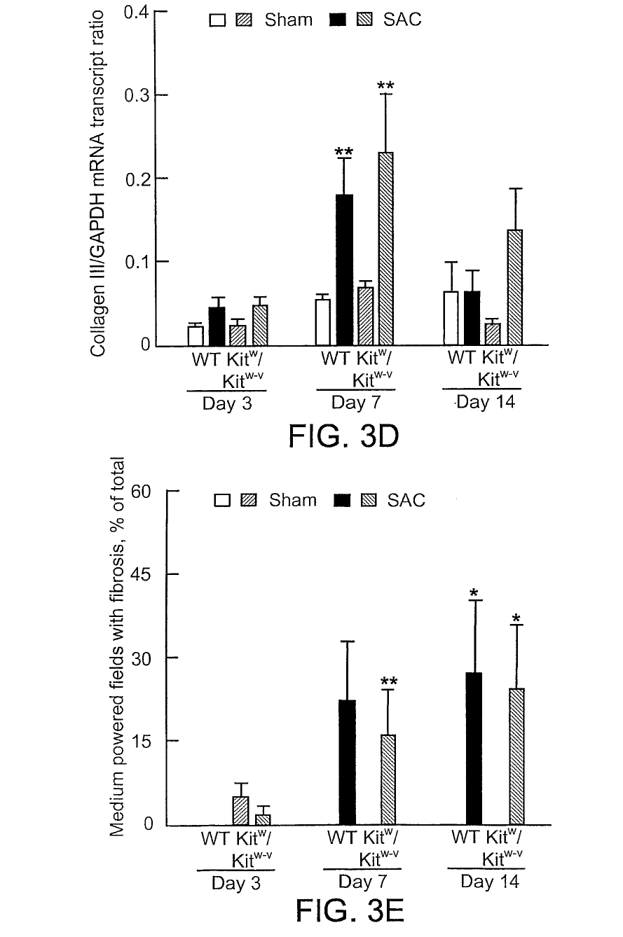

To address the issue of cell proliferation in the Kit^{w}/Kit^{w-v}-SAC hearts, directly, DNA synthesis and cell cycling were examined in cardiomyocytes, BrdU labeling and expression of Ki67 in the nuclei of both interstitial cells (non-cardiomyocytes) and cardiomyocytes were assessed, the latter identified by their expression of cardiac myosin heavy chain. Cardiomyocytes were also evaluated for nuclear phosphorylated histone-3 (H3P), since it is associated with chromosomal condensation that accompanies the onset of mitosis. In both genotypes SAC resulted in a 10-fold increase in the number of BrdU⁺ LV interstitial cells at 7-days, as well as increased fibrosis and collagen I and III mRNA expression (FIG. 3). These findings are consistent with cardiac fibroblasts forming a large proportion of interstitial cells. Cardiac fibroblasts were also identified by immunostaining for the intermediate filament protein, vimentin, in both the WT-SAC and Kit^{w}/Kit^{w-v}-SAC animals (e.g., FIG. 3B).

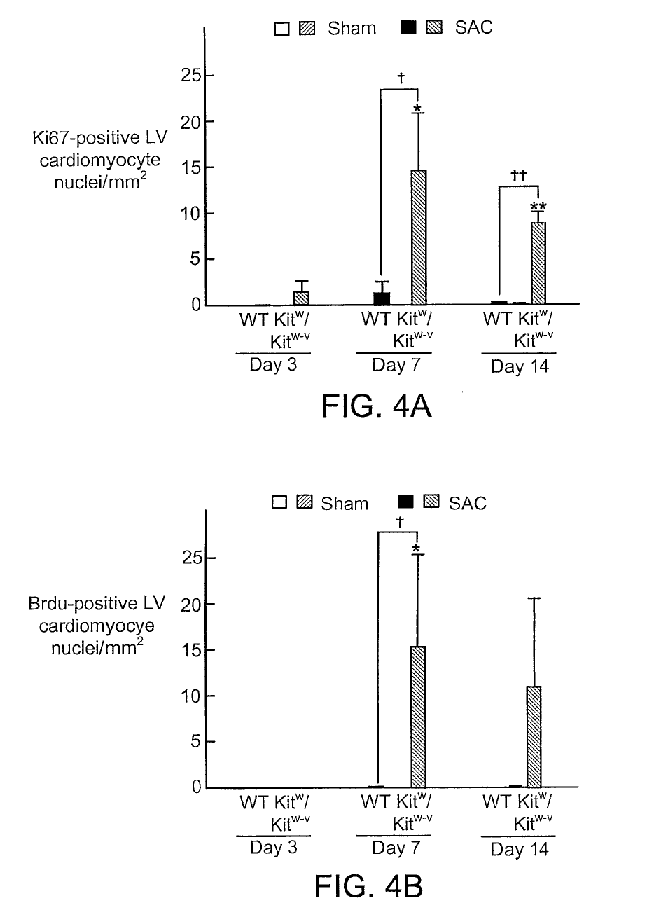

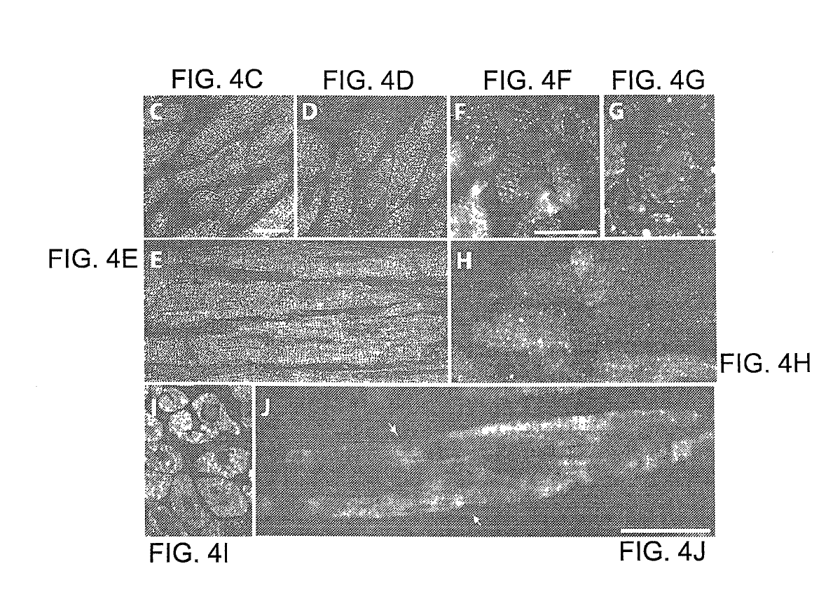

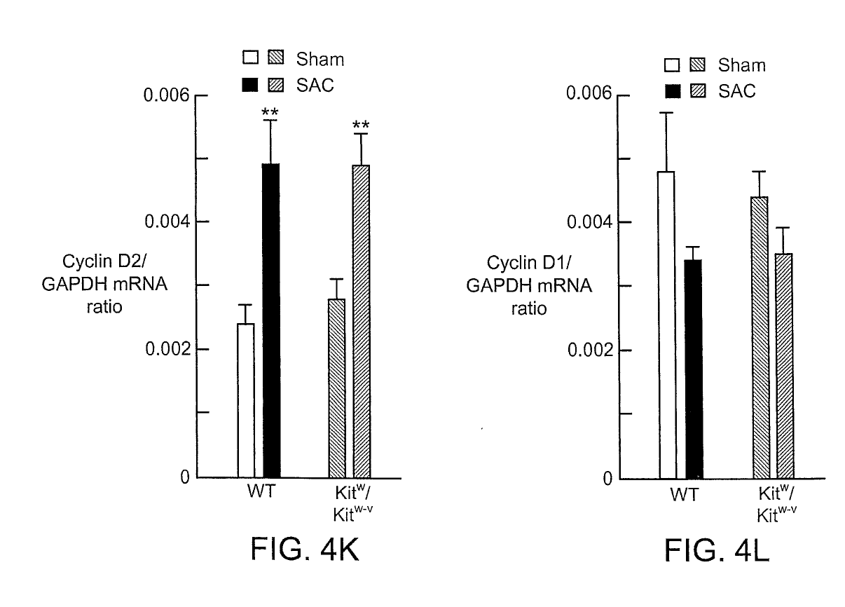

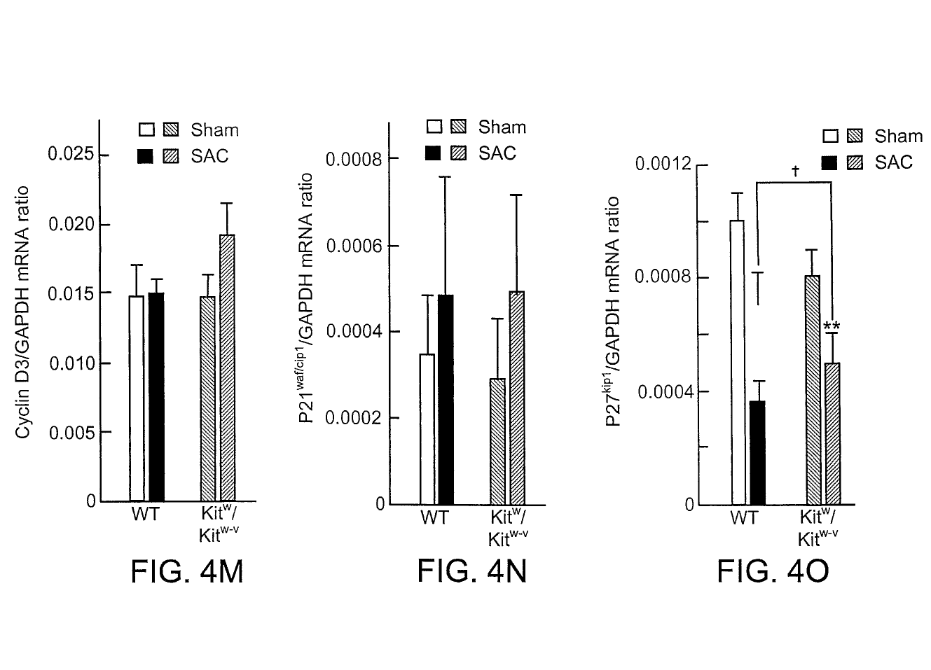

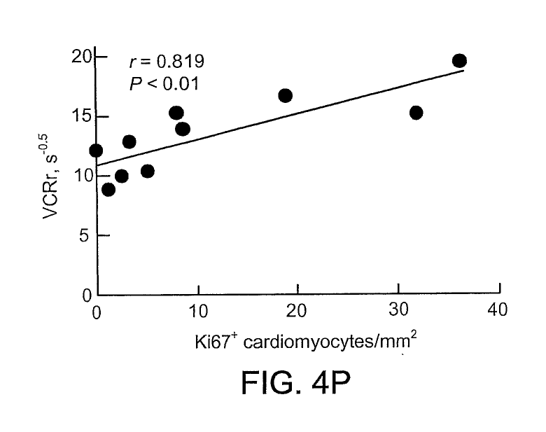

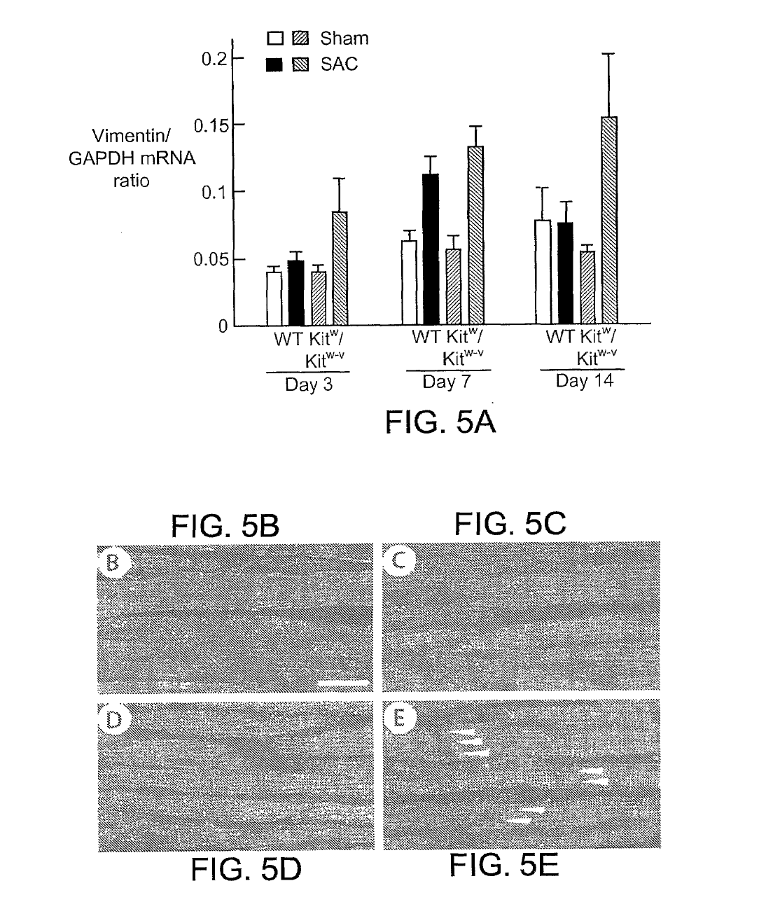

In the absence of SAC, no BrdU⁺ or Ki67⁺ cardiomyocytes were observed in multiple LV sections of WT or Kit^{w}/Kit^{w-v} mice (FIGS. 4, A and B)—that is, under basal conditions less than 0.001% of total cardiomyoeytes were dividing in WT or Kit^{w}/Kit^{w-v} mice (calculated from FIGS. 4, A and B and FIG. 2E). Similarly, no BrdU⁺ and only two Ki67⁺ nuciei/mm² were observed at 3 to 14 days of SAC in the WT mice. However, both BrdU³⁰ and Ki67⁺ cardiomyocytes were readily apparent in Kit^{w}/Kit^{w-v}-SAC hearts, particularly at 7 and 14 days of SAC (FIG. 4, A-H). For example, at day 7 after SAC, cardiomyocytes that were dividing (as assessed by nuclear BrdU- or Ki67-labeling) had increased to approximately 2% of the total in Kit^{w}Kit^{w-v} hearts (calculated from FIGS. 4, A and B and FIG. 2E). Moreover, there was a tight positive correlation between BrdU⁺ and Ki67⁺ cardiomyocyte density in Kit^{w}/Kit^{w-v} mouse hearts at 7 and 14 days after SAC (r=0.96, P<0.001). Many BrdU⁺ cardiomyocytes were also H3P⁺ (FIGS. 4, I and J). Overlay of cardiomyocytes by BrdU⁺ or Ki67⁺ interstitial cells or by mobilized extra-cardiac cells, which could create the appearance of a proliferating cardiomyocyte, was excluded by confocal laser scanning microscopy or by digital deconvolution. Importantly, cardiomyocytes in which nuclei were BrdU⁺ (FIG. 4E), Ki67⁺ (FIG. 4H) and/or H3P⁺ (FIG. 4J) were large, rod-shaped cells with mature sarcomere organization, and together with adjacent mature cardiomyocytes appeared to form an integrated myofiber. This contrasts with the nests of small, round, spindle-shaped cells lacking sarcomeres observed with the apparent, transdifferentiation of hemopoietic stem cells (HSCs) into cardiomyocytes after their injection into myocardium (D. Orlic et al., (2001)), and with the markedly smaller (over one order of magnitude) apparent cardiomyocytes that result from differentiation of cultured resident cardiac stem cells (CSCs) (D. Orlic et al., (2001)). Cardiomyocyte vimentin expression was also observed throughout the LV in the region of the intercalated discs, in Kit^{w}/Kit^{w-v} but not WT mice, at day 14 after SAC (FIG. 5). Although vimentin is expressed abundantly by fetal cardiomyocytes, after birth its expression is limited to fibroblasts, even in the setting of cardiac failure (S. Di Somma et al., (2000)). Its reactivation in the Kit^{w}/Kit^{w-v}-SAC animals, therefore, is consistent not with fetal gene re-programming as observed in hypertrophy, but rather with a cardiomyocyte regenerative response.

Mast cell (MC) deficiency is a prominent phenotype in Kit^{w}/Kit^{w-v} mice (S. J. Galli, Y. Kitamura, (1987)), and MC stabilization in rats attenuates perivascular cardiac fibrosis due to chronic hypertension (B. Hocher et al., (2002)). But, MCs are relatively rare in the WT mouse LV (2.2±0.64 MCs/mm², n=5) and did not increase after 7 days of SAC (1.8±0.37 MCs/mm², n=5). Moreover, despite MC deficiency in the Kit^{w}/Kit^{w-v} mice, proliferation of cardiac interstitial cells, collagen I and III expression, and the degree of cardiac fibrosis were similar in Kit^{w}/Kit^{w-v}-SAC and WT-SAC mice (FIG. 3). Cromolyn blocks MC-dependent phenomena (R. Chen et al., (2001)). Although treatment of WT mice with cromolyn (60 mg/kg/day) reduced MC density by approximately 80% (to 0.45±0.2 MCs/mm², n=4), it did not alter the response to SAC-LV function and cardiomyocyte hypertrophy were similar in cromolyn-treated and vehicle-treated WTSAC mice; hyperplasia and BrdU⁺ or Ki67⁺ cardiomyocytes were not evident.

To further explore the mechanism of cardiomyocyte hyperplasia, expression of cell cycle regulators was evaluated using quantitative real-time RT-PCR of LV myocardial mRNA from WT and Kit^{w}/Kit^{w-v} mice. Cyclins, activators of cyclin-dependent kinases (CDKs), play an important role in the commitment to cell division (T. Hunter, et al. (1994)). CDK4 and the Dtype cyclins facilitate transit through the cell cycle restriction point, and targeted overexpression of D-type cyclins increases cardiomyocyte DNA synthesis (K. B. S. Pasumarthi, et al. (2005)). At day 7 of SAC-induetion, when cardiomyocyte proliferation was most evident in Kit^{w}/Kit^{w-v}-SAC mice (FIGS. 4, A and B), expression of cyclin D1 was increased (FIG. 4K). Although significant, this change occurred in both WT- and Kit^{w}/Kit^{w-v}-SAC mice. Cyclins D2 (FIG. 4L) and D3 (FIG. 4M) were unchanged. In contrast to these cyclin responses, which were concordant in the two mouse genotypes, expression of the CDK inhibitor, p27^{kip1}, but not p21^{waf1/cip1}, fell to a level 35% lower (P<0.05) in Kit^{w}/Kit^{w-v}-SAC mice than in WT-SAC mice (FIGS. 4, N and O). The anti-proliferative effects of p27^{kip1} are dose-dependent (R. A. Poolman, et al. (1999)). Moreover, loss of cardiomyocyte proliferation after birth coincides with increased p27^{kip1} expression (K. B. S. Pasumarthi, L. J. Field (2002)), while neonatal cardiomyocytes display reduced p27^{kip1}-expression when induced to proliferate in response to FGF1 and p38α MAP kinase-inhibition (F. B. Engel. et al., (2005)). Thus, reduction in p27^{kip1} expression in the Kit^{w}/Kit^{w-v}-SAC mice, at a time when cyclin D1 expression had increased (FIG. 4K), is consistent with a cardiomyocyte regenerative response and re-entry of cardiomyocytes into the cell cycle. Thus, adult mammalian cardiomyocytes can divide in vivo. The effects of mutational inactivation of c-Kit are not due to MC deficiency but involve inhibition of a pathway that is inhibitory to cell cycle re-entry, and/or activation of a pro-proliferative pathway—albeit only when instigated by a growth stimulus, such as hypertension. This restricted proliferative response contrasts with the basal hyperplasia observed with overexpression of c-myc (T. Jackson et al., (1991)), telomerase (H. Oh et al., (2001)) or cyclin D1, D2 and D3 (K. B. S. Pasumarthi et al. (2005)), or with inactivation of p27^{kip1} (R. A. Poolman, et al. (1999)), demonstrating that it is possible to selectively activate cardiomyocyte DNA synthesis under conditions of myocardial stress. Also important is that re-acquisition of cardiomyocyte proliferative-responsiveness in the Kit^{w}/Kit^{w-v} mice after SAC appears to be functionally significant. This is evident from the strong positive relation between the number of both Ki67⁺ (FIG. 4P) and BrdU⁺ cardiomyocytes (r=0.73, P<0.05) and cardiac contractility. Thus, cardiomyocyte hyperplasia in Kit^{w}/Kit^{w-v}-SAC mice appears to contribute to increased contractile function and reduced mortality.

### Example 2

### c-Kit Tyrosine Kinase Dysfunction Increases Hypertension-Dependent Expansion of c-Kit+ Cardiac Stem Cells

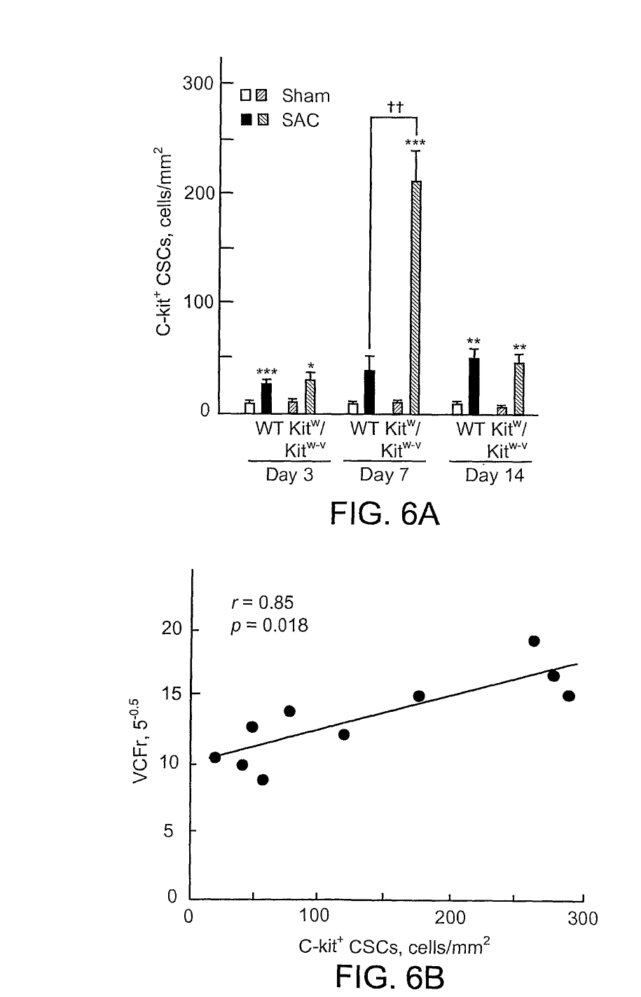

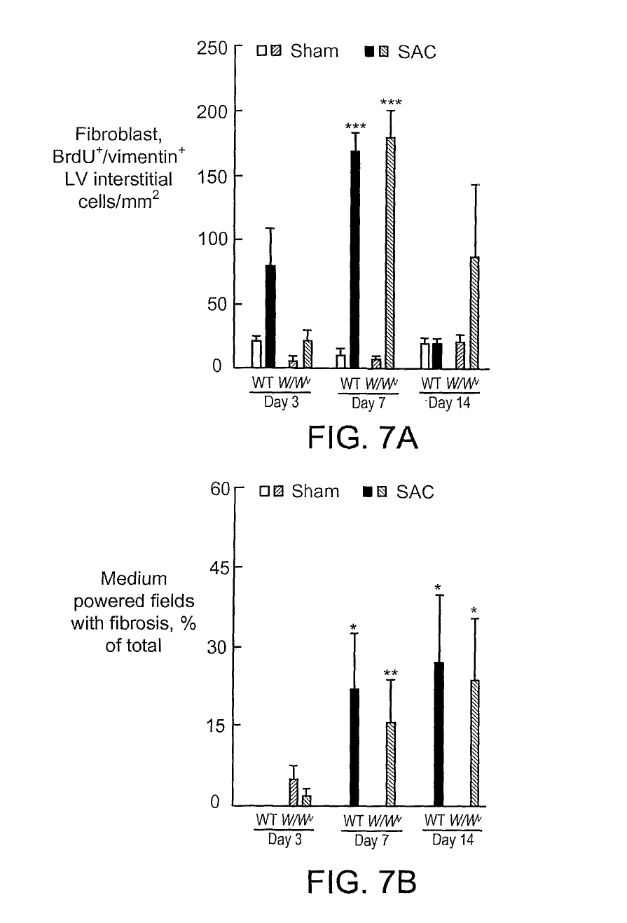

Unlike terminally differentiated cardiac cells, cardiac stem cells (CSCs) are small cells that do not express mature cardiac markers and can proliferate. There are several different but overlapping types of CSCs, which are grouped according to cell surface markers, e.g., ckit+, Sca1+, MDR1+, isl1+, e-kit+ CSCs differentiated into cardiomyocytes contributing to repair of a damaged heart. CSCs were identified in LV mid-wall tissue sections by their small size (10-20 μm diameter), by immunohistochemical localization of stem cell surface markers c-kit, Sca-1, or MDR1, and by the absence of the hematopoietic stem cell marker CD45. c-kit⁺ CSC numbers in the LV of sham-operated WT or W/W^{v} mice were generally low (˜10 CSCs/mm²), but Sca-1⁺ CSC numbers were lower (<0.1 CSCs/mm²) and MDR1⁺ CSCs were not observed. In the W/W^{v}- and WT-SAC LV myocardium, c-kit⁺ CSCs occurred individually, in pairs or in large clusters. c-kit⁺ CSC clusters were not seen in the LVs of WT- or W/W^{v}-sham mice and were rare in WT-SAC mice. Compared to sham controls, 7 days of SAC increased c-kit⁺ CSCs ˜19-fold in the W/W^{v} LV (P<0.001; FIG. 6A). Compared to the WT-SAC LV, the increase was ˜5.5-fold (P<0.01; FIG. 6A). Hypertension and/or c-kit dysfunction did not affect Sca-1⁺ or MDR-1³⁰ CSC levels. Mast cells also express c-kit, and W/W^{v} mice are mast cell deficient. The effect of c-kit dysfunction on CSCs is likely to be direct, however, because, in 7-day-SAC WT mice, suppression of mast cell degranulation with cromolyn (60 mg/kg/day; started 7 days before SAC) did not significantly increase c-kit⁺ CSCs (38±3 CSCs/mm², n=5), relative to vehicle controls (31±2 CSCs/mm², n=5). In 7-day-SAC LVs, fibroblast proliferation (vimentin⁺/BrdU⁺ interstitial cells⁹) increased ˜10-fold over sham LVs in both genotypes; thus, this proliferation was independent of c-kit dysfunction (FIG. 7A). Moreover, extracellular matrix deposition was similar in WT- and W/W^{v}-SAC mice (FIG. 7B), and cardiomyocyte apoptosis was not observed in either genotype. Capillary densities were also similar in 7 day-SAC WT and W/W^{v} mice (1,610±37 endothelial cells/mm² in WT-SAC LV versus 1,750±73 endothelial cells/mm² in W/W^{v}-SAC LV; n=6/group). Collectively, these findings indicate that c-kit⁺ CSC expansion is induced by hypertension and selectively increased by c-kit dysfunction.

### Example 3

### c-kit Protein Expression in Cardiomyocytes Adjacent to Large c-kit⁺ Cardiac Stern Cell (CSC) Clusters

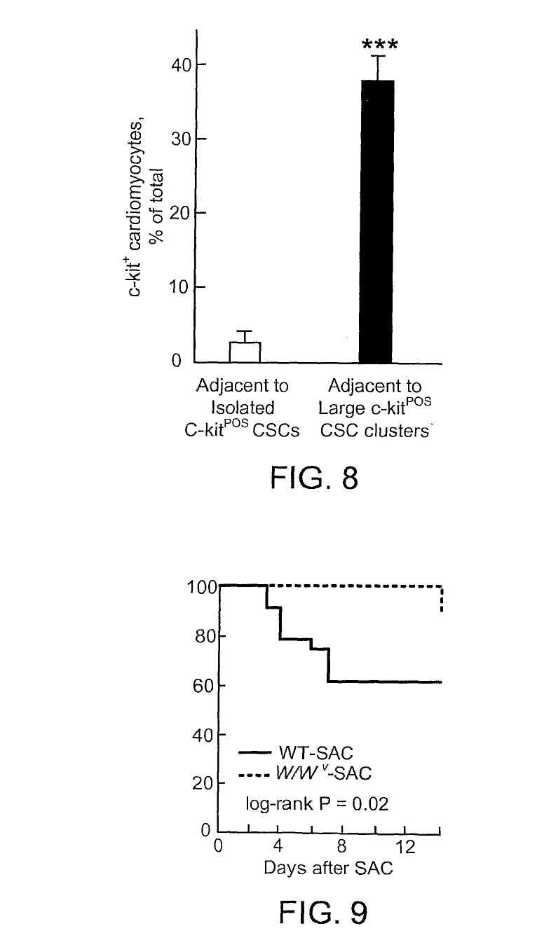

To determine whether proliferating cardiomyocytes are derived from c-kit⁺ CSCs, expression of c-kit in cardiomyocytes adjacent to c-kit+ CSC clusters was examined. Endogenous c-kit⁺ CSCs, unlike donor CSCs, cannot be labeled in situ. Expression of c-kit in cardiomyocytes adjacent to c-kit⁺ CSC clusters might be expected if they were derived from c-kit⁺ CSCs, c-kit is not seen in WT cardiomyocytes but is abundant in CSCs, c-kit⁺ cardiomyocytes were observed adjacent to clusters of c-kit⁺ CSCs, but the frequency of these cells was related to the size of the cluster; ˜17-fold more c-kit⁺ cardiomyocytes were observed adjacent to large c-kit⁺ CSC-clusters than adjacent to isolated (1-2 cells) c-kit⁺ CSCs (P<0.001). Without being bound by theory, this CSC-dependent acquisition of a CSC phenotype by cardiomyocytes suggests fusion of the CSC with the cardiomyocyte, rather than differentiation of the CSC, because c-kit expression is lost when CSCs differentiate into mature cardiomyocytes. Furthermore, while CSC differentiation leads to the formation of GATA-4⁺ cardiomyocyte progenitors, only 0.23±0.15% of c-kit⁺ CSCs in W/W^{v}-7-day-SAC mice (n=5) were GATA-4⁺. Taken together, the positive association between c-kit⁺-CSCs and Ki67⁺-cardiomyocytes in W/W^{v} mice after 7-14 days of SAC (r=0.689, P<0.02), and direct evidence of cardiomyocyte cell-cycle reentry in c-kit⁺ cardiomyocytes (FIG. 8), are most consistent, with c-kit⁺-CSC expansion in proliferative W/W^{v} LV niches producing fusion-driven cardiomyocyte proliferation, although dedifferentiation of cardiomyocytes is possible. Adult cardiomyocytes do not proliferate for reasons that include their lack of telomerase activity¹². But c-kit⁺ CSCs have abundant telomerase activity. Fusion or dedifferentiation could increase telomerase activity in cardiomyocytes causing them to reenter the cell cycle.

Alternatively, c-kit⁺ CSC-derived cytokines could also cause neighboring cardiomyocytes to reenter the cell cycle by adopting a more primitive state. To test this, gene profiles of WT and W/W^{v} mice LVs after 7 days of SAC or sham operations using cDNA maicroarrays were determined. Gene profiles of WT-sham and SAC mice and W/W^{v}-sham and -SAC mice analyzed by Ingenuity Pathway Analysis identified increases in several cytokines. Those genes whose expression were selectively increased in W/W^{v}-SAC mice over sham-operated mice, but not in WT-SAC mice relative to its sham-operated control, included insulin-like growth factor-1, interlukin-6, bone morphogenic protein-1, and chemokine (C-C motif) ligand 2 (CCL2CCL2. These soluble cytokines can potentially cause the cardiomyocytes to adopt the phenotype of a more primitive state (i.e., dedifferentiate). For example, the cytokines could induce expression of c-kit⁺ and cell cycle reentry. Some cytokines, for example, insulin-like growth factor-1 improve cardiac function.

Further evidence suggests that CSC expansion and cardiomyocyte proliferation have functional consequences in W/W^{v}-SAC mice. In WT mice, SAC produces an increase in LV mass that is accompanied by an increase in cardiomyocyte cross-sectional area (LV enlargement through cardiomyocyte hypertrophy), but in W/W^{v}-SAC mice there-is a similar increase in LV mass with a markedly smaller change in cardiomyocyte cross-sectional area (P<0.01). Early mortality increases after severe acute pressure overload, and hypereontractile cardiac function reduces this mortality. Therefore, it was determined if improved cardiac contractility after hypertension could improve survival in W/W^{v}-SAC mice. In the first 7 days of hypertension, 41% of the WT died but no W/W^{v} mice (P<0.05; FIG. 9). The early response to hypertension is adaptive LV growth⁵. In W/W^{v} mice, remodeling with SAC was not merely adaptive, since it increased LV contractility (VCFr) more than in normotensive W/W^{v} mice (Table 3, above). The enlarged LVs in hypertensive WT and W/W^{v} mice had similar LV capillary densities to those seen in the smaller LVs of their normotensive controls, indicating that neovascularization is adaptive and matches the growth of the LV (see Example 4 below). Improvement in LV contractility in W/W^{v}-SAC mice may therefore result from an increase in LV muscle mass, but without the metabolic penalty that results from cardiomyocyte hypertrophy. A significant positive correlation between c-kit⁺ CSCs and VCFr in W/W^{v} mice after 7 and 14 days of SAC (P<0.02; FIG. 6B) further suggests that an in vivo expansion of c-kit⁺-CSCs improves systolic function in hypertensive mice.

A critical balance between proliferative signals and apoptotic signals is important for cell proliferation. The c-kit tyrosine kinase domain stimulates both proliferative and apoptotic signals in a cell specific manner. This dual phenotype of c-kit is shared with other type III receptor tyrosine kinases, since the platelet-derived growth factor receptor can also induce apoptosis. As described above, c-kit tyrosine kinase dysfunction causes stress-induced CSC expansion in vivo, with associated cardiomyocyte proliferation and improvement of systolic function. CSC differentiation or CSC-cardiomyocyte fusion could be the pathway for cardiomyocyte proliferation. The identification of c-kit tyrosine kinase as a regulator of CSC proliferation provides a promising target for therapeutic interventions to promote CSC-driven cardiomyocyte proliferation in chronic hypertensive heart disease.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which wilt be limited only by the appended claims.

Those skilled in the art will recognize, or be able to ascertain using no more than routine experimentation, many equivalents to the specific embodiments of the method and compositions described herein. Such equivalents are intended to be encompassed by the claims.

### REFERENCES

Chen R, et al., J. Clin. Invest. 108, 1151 (2001).
Di Somma S, et al., Heart 84, 659 (2000).
Du X-J, et al., J. Mol. Cell, Cardiol. 37, 979 (2004).
Engel F B, et al., Genes Dev. 19, 1175 (2005).
Frustaci A, et al., Circ. Res. 87, 1123 (2000).
Galli S J., et al., Am. J. Pathol. 127, 191 (1987).
Galli S J., et al., Am. J. Pathol. 127, 191 (1987).
H, Oh et al., Proc. Natl. Acad. Set. U.S.A.98, 10308 (2001).
Hara M, et al., J. Exp. Med. 195, 375 (2002).
Hilfiker-Kleiner D, et al., Circulation, 112, 1470 (2005).
Hocher B, et al,. J. Hypertens. 20, 745 (2002).
Hunter T., et al., Cell 79, 573 (1994).
Hyink D P, et al. Am. J. Physiol. 270, F886 (1996).
Jackson T, et al., Mol. Cell. Biochem. 104, 15 (1991).
Li M, et al., J. Clin. Invest. 114, 112 (2004).
Orlic D, et al. Nature 410, 701 (2001a),
Orlic D, et al., Proc. Natl. Acad. Sci. USA 98, 10344 (2001b).
Pasumarthi K B S., et al., Circ. Res. 96, 110 (2005).
Pasumarthi, K B S, et al., Circ. Res. 90, 1044 (2002).
Perry G J, et al., Circulation 103, 1012 (2001).
Poolman R A, et al., Circ. Res. 85, 117 (1999).
Vatner D E, et al., Circ. Res. 14, 802 (2000).
Wakasugi K, et al., J. Biol Chem. 277, 20124 (2002).

1. A method of inhibiting hypertension-induced hypertrophy of cardiac cells, comprising contacting cardiac cells with an inhibitor of c-Kit activity.

2. A method of reducing or inhibiting hypertension-induced hypertrophy of cardiac cells in a subject, comprising administering to the subject a therapeutic amount of an inhibitor of c-Kit activity.

3. The method of claim 1, wherein the inhibitor is Imatinib mesylate or an analog or derivative of Imatinib mesylate.

4. The method of claim 1, wherein the inhibitor is SU5416, SU6668, or an analog or derivative of SU5416 or SU6668.

5. The method of claim 1, wherein the inhibitor is a functional nucleic acid.

6. The method of claim 1, wherein the inhibitor blocks the binding of stem cell factor (SCF) to c-Kit.

7. The method of claim 6, wherein the inhibitor is an antibody.

8. The method of claim 7, wherein the antibody is specific for c-Kit.

9. The method of claim 7, wherein the antibody is specific for SCF.

10. The method of claim 6, wherein the inhibitor is a soluble c-Kit receptor.

11. The method of claim 1, wherein the inhibitor of c-Kit activity induces cardiac cell proliferation.

12. The method of claim 1, wherein the inhibitor of c-Kit activity improves contractility of the cardiac cells.

13. The method of claim 2, where the subject is hypertensive.

14. A method of screening for agents that inhibit hypertension-induced hypertrophy of cardiac cells, comprising:
a. contacting a cardiac stem cell with the agent to be tested, and
b. measuring c-Kit activity, wherein a decrease in c-Kit activity as compared to a control indicates that the agent inhibits hypertension-induced hypertrophy of the cardiac cells.

15. A method of increasing cardiac stem cell numbers, comprising contacting a cardiac stem cell with an inhibitor of c-kit activity.

16. A method of identifying cytokines associated with inhibition of hypertension-induced hypertrophy, comprising
a. contacting cardiac cells with an inhibitor of c-kit activity; and
b. detecting changes in cytokine expression or activity, wherein an increase in cytokine expression or activity as compared to control indicates that the cytokine is associated with inhibition of hypertension-induced hypertrophy.

17. A method of inhibiting hypertension-induced hypertrophy in a subject, comprising administering to the subject a cytokine identified by the method of claim 16.

18. A method of increasing cardiac cell numbers, comprising contacting a cardiac cell with a cytokine identified by the method of claim 16.

19. The method of claim 17, wherein the cytokine is selected from the group consisting of insulin-like growth factor-1, interlukin-6, bone morphogenic protein-1, and chemokine (C-C motif) ligand 2 (CCL2).

20. The method of claim 2, wherein the inhibitor is Imatinib mesylate or an analog or derivative of Imatinib mesylate.

21. The method of claim 2, wherein the inhibitor is SU5416, SU6668, or an analog or derivative of SU5416 or SU6668.

22. The method of claim 2, wherein the inhibitor is a functional nucleic acid.

23. The method of claim 2, wherein the inhibitor blocks the binding of stem cell factor (SCF) to c-Kit.

24. The method of claim 2, wherein the inhibitor of c-Kit activity induces cardiac cell proliferation.

25. The method of claim 2, wherein the inhibitor of c-Kit activity improves contractility of the cardiac cells.
